(19) United States
(12) Patent Application Publication
(10) Pub. No.: US 2007/0212333 A1
(43) Pub. Date: Sep. 13, 2007

(54) Methods and compositions for the treatment of dystonia

(75) Inventors: Yuqing Li, Vestavia Hills, AL (US); Mai Dang, Champaign, IL (US); Fumiaki Yokoi, Birmingham, AL (US)

Correspondence Address: MARSHALL, GERSTEIN & BORUN LLP, 233 S. WACKER DRIVE, SUITE 6300, SEARS TOWER, CHICAGO, IL, 60606, US

(73) Assignee: UNIVERSITY OF ILLINOIS URBANA, Urbana, IL (US)

(21) Appl. No.: 11/544,070

(22) Filed: Oct. 5, 2006

Related U.S. Application Data
(60) Provisional application No. 60/724,925.
(60) Provisional application No. 60/725,394, filed on Oct. 11, 2005.

(51) Int. Cl. A61K 48/00; C12N 15/861

(52) U.S. Cl. 424/93.2; 435/456; 514/44

(57) ABSTRACT

The present invention relates to methods and compositions for the treatment of dystonia in a mammal. More particularly the methods of the invention involves decreasing the expression of wild-type Dyt1 in the Purkinje cells of mammals exhibiting symptoms of dystonia in order to treat the dystonia.

This application claims the benefit of priority of U.S. provisional application No. 60/725,394 which as filed Oct. 11, 2006 as well as U.S. provisional application No. 60/724,925 which was filed Oct. 7, 2006. The text of each of these prior applications is specifically incorporated herein by reference.

This invention was made with government support under grant T32 GM007143 awarded by the National Institutes of Health. The government has certain rights in the invention.

### FIELD OF THE INVENTION

The present invention relates to methods and compositions for the treatment of dystonia in a mammal.

### BACKGROUND OF THE RELATED ART

Dystonia is defined as sustained muscle contractions that cause twisting and repetitive movements or abnormal postures. The abnormal movements are involuntary and sometimes painful. Dystonia is generally believed not to be a muscle disease and instead is caused by deficits in the brain. The patients usually have normal intelligence and no associated psychiatric disorders except for an increased risk of recurrent major depression [2]. Dystonia is also classified as a neurochemical disorder where no degeneration of neurons is observed. Patients with dystonia have no signs of gross anatomical changes in the brain. Dystonia can be classified according to the body parts they affect. Generalized dystonia affects most or all of the body. Focal dystonia is localized to a specific part of the body. Multifocal dystonia involves two or more unrelated body parts. Segmental dystonia affects two or more adjacent parts of the body. Hemidystonia involves the arm and leg on the same side of the body. Oppenheim dystonia (DYT1 dystonia) is a generalized, early-onset form of dystonia. Symptoms usually appear in childhood or adolescence, first affecting a limb and eventually traveling to other body parts. In many cases, all parts of the body are eventually affected with the cranial muscles mostly spared [3]. Affected individuals could be seriously disabled and confined to a wheelchair.

The inheritance of DYT1 dystonia has been determined to be autosomal dominant with a penetrance of 30-40% [4]. DYTI carriers who do not display symptoms by age 28 are very unlikely to ever develop dystonia [3]. The DYTI locus was mapped to chromosome 9q34.1 and subsequently cloned [5]. The mutation in human DYT1 gene appears to be a 3-bp deletion (GAG) in the coding region of DYT1 (DYT1^{ΔGAG}). The 3-bp mutant deletes a glutamic acid residue in the C-terminal coding region [6]. This 3-bp deletion in the heterozygous state occurs in 50-60% of non-Jewish and over 90% of Ashkenazi Jewish early-onset dystonic patients [7]. Furthermore, DYT1 AGAG is related not only to generalized dystonia, but also to some forms of focal or multifocal dystonia [8,9]. There is also a single case of an 18-bp deletion reported in one patient [10] in combination with a mutation of ε-sarcoglycan gene [11], which is implicated in causing myoclonus-dystonia [12].

DYT1 codes for torsinA, a novel member of the AAA+ superfamily (ATPase Associated with a variety of cellular Activities), which includes ATPases involved in protein chaperone functions, vesicle trafficking, and membrane fusion [6]. TorsinA also has an ATPase activity [13]. It is widely expressed in the body and is seen in many brain regions [14-18]. Protein and mRNA studies show torsinA localization in high quantities to the cerebellar Purkinje cells, cortical layers III and V, and the hippocampus. TorsinA is present in the dopaminergic system and in the striatum. Among neurons in the striatum, cholinergic interneurons of the caudate putamen show the highest level of torsinA expression. TorsinA is present in cell bodies as well as ineurites [15,17,19].

Biochemical studies have been reported concerning the properties of torsinA and torsinA^{ΔE}. Recombinant torsinA and torsinA E are membrane-associated glycoproteins that required detergents for solubilization and purification. Both forms of torsinA display ATPase activity with similar kinetic values. Collectively, these data reveal that torsinA is a membrane-associated ATPase and indicate that the torsinA^{ΔE} does not cause gross changes in its catalytic or structural properties [20]. Like wild-type torsinA, torsinA^{ΔE} displays cleavage of the 20 N-terminal amino acids signal peptide and membrane association region (amino acids 24 to 40). However, torsinA^{ΔE} is unlike the wild-type torsinA in that the mutated form was not secreted in the S2 culture even after deletion of the membrane-anchoring segment. This molecular behavior indicates that the torsinA^{ΔE} has a structurally distinct, possibly misfolded form of torsinA, which cannot be properly processed in the secretory pathway of eukaryotic cells [21]. This finding suggests torsinA^{ΔE} is a loss-of-function mutation. Recently, through the use of a yeast two-hybrid system, the light chain subunit of kinesin-I has been identified as an interacting partner for torsinA [22]. In cultured cortical neurons, both proteins co-localize along neuronal processes with enrichment at growth cones. Wild-type torsinA co-localize with endogenous KLC1 at the distal end of processes, whereas mutant torsinA remain confined to the cell body. These studies suggest that wild-type torsinA undergoes anterograde transport along microtubules mediated by kinesin and may act as a molecular chaperone regulating kinesin activity and/or cargo binding.

Although the function of torsinA is largely unknown, overexpression studies in cell culture have offered clues to its potential intracellular function. The overexpression of wild type torsinA protein protected cells from toxicity. Overexpression of worm torsin protein suppressed polyglutamine-induced protein aggregation [23]. Studies have also suggested that torsinA is involved in the stress response. Cellular transfection studies of wild-type and mutant DYT1 showed that overexpressed normal torsinA co-localized with stress proteins BiP and PDI [24,25]. TorsinA also has been suggested as playing a role in correcting aggregated protein clusters. When overexpressed in cells, torsinA, like several heat shock proteins, disaggregated alpha-synuclein clusters [26]. This activity was lost in torsinA^{ΔE}. These findings again suggest that torsinA^{ΔE} is a loss-of-function mutation. Another study showed that an overexpressed level of torsinA provided protection for COS-1 and PC12 cells against oxidative stress. An abundance of torsinA reduced cell death caused by H₂O₂ exposure [27]. Overexpression of torsinA E also appears to move the torsinA^{ΔE} proteins to nuclear envelope [28-32].

Neurochemical analyses of human brain tissues and hemidystonic primates show that the dopaminergic system may be involved in dystonia. Expression analysis has revealed that DYT1 mRNA is present at high levels in the pars compacta of the substantia nigra [14,15]. The greater than normal ratio of dopamine metabolite 3,4-dihydroxyphenylacetic acid to dopamine in tissues from dystonic patients suggests dopamine metabolism is increased [33]. Primates treated with MPTP injections that eventually develop Parkinson's, but first display symptoms of dystonia for a short period of time were found in positron emission tomography studies to have a decrease in D2 binding in the putamen [34]. Recently, decreased striatal D2 receptor binding in non-manifesting carriers of the DYT1 dystonia mutation has been reported [35]. Overexpression of wild and mutant torsinA in cultured human cell lines has revealed a potential pathological role for mutant torsinA in cellular trafficking of the dopamine transporter [36] as well as dopamine release [37]. Whether or not these abnormalities are really correlated and the manner in which they are correlated to DYT1 dystonia is still unknown. Nevertheless, they do suggest that in contrast to neurodegeneration seen in Parkinson's patients, an intact but abnormally functioning dopaminergic system may be involved in dystonia.

Animal models of neurological diseases are extremely useful for understanding the pathophysiology of the disorder and for developing effective therapeutic treatments. Past attempts using experimentally induced dystonia in animals [38] and genetic animal models [39] with natural spontaneous mutations have provided clues about the nature of the disorder. These models have contributed substantially to the general understanding of the pathophysiology of dystonia. However, these animal models have limitations in identifying the primary defects of dystonia since the lesions produced or genes mutated in these animal models are different from those altered in human patients. Cloning of the DYT1 gene in humans and subsequent identification of the mouse homolog [40] made it possible to use reverse genetics directly to create animal models of DYT1 primary dystonia. Various groups have reported the creation of transgenic lines that overexpress human mutant torsinA that show motor deficits [41,42]. A more suitable model for DYT1 dystonia is one that replicates the torsinA expression pattern and level seen in patients. In addition, the study of any protein's function can greatly benefit from the creation of a mouse line in which the expression of the interested protein is abolished.

While much has been learned about dystonia through the above studies, it is still generally understood in the art that dystonia occurs as a result of expression of a mutant Dyt1 and that it is desirable to increase the expression of wild-type of TorsinA in dystonia patients. Nevertheless there remains a need to identify additional therapies for dystonia.

### SUMMARY OF THE INVENTION

The present invention is based on the surprising finding that Purkinje cell-specific Dyt1 inactivation improves motor performance in dystonia mice. As such, in contrast to the findings of the prior art, the present invention is directed to alleviating the symptoms of dystonia and dystonia-related disorders in mammals by selectively inactivating Dyt1 in the Purkinje cells of mammals that exhibit such symptoms of dystonia.

In one embodiment, the invention provides for methods of treating a neuronal disease in a mammal comprising selectively down-regulating the expression and/or activity of wild type Dyt1 in Purkinje cells of said mammal. The invention particularly provides for methods of treating human subjects having a neuronal disease.

In some methods of the invention, selectively down-regulating wild-type Dyt1 in Purkinje cells comprises administering to the mammal an expression construct that comprises a Purkinje cell-specific promoter operably linked to a nucleic acid that inhibits the expression of wild-type Dyt1. The expression construct administered to the mammal may be a viral vector such as an adenoassociated viral vector.

In certain embodiments, the viral vector administered to a mammal in the methods of the invention comprises a polynucleotide sequence of about 8 to 80 nucleotides in length targeted to a nucleic acid molecule encoding DYT1, wherein said polynucleotide of 8 to 80 nucleotides specifically hybridizes in Purkinje cells with a nucleic acid molecule the encodes DYT1 and inhibits the expression of DYT1 in said Purkinje cells. In particular, the invention contemplates methods of administering viral vectors comprising polynucleotide sequences of about 15 to about 30 nucleotides in length. The invention also contemplates administering viral vectors comprising polynucleotide sequence of about 20 to about 25 nucleotides in length.

In another embodiment, the invention provides for administering an expression construct that selectively down-regulates the expression and/or activity of wild-type Dyt1 in Purkinje cells to a mammal. In some methods of the invention, the expression construct is administered systemically to the mammal. In other methods of the invention, the expression construct is administered via an intrathecal catheter. In invention also provides for methods wherein the expression construct is administered via intracerebellar injection.

The invention also provides for carrying out any of the preceding methods wherein the expression construct is administered in combination with at least one additional drug that is used for the treatment of dystonia or related tremor disorders. The Dystonia Medical Research Foundation ( http://www.dystonia-foundation.org/treatment/oral.asp.) notes that various categories of drugs may be used to treat dystonia. It is noted that medications that lessen the symptoms of pain, spasm, and abnormal posturing and function will be particularly useful. As these treatments have differing mechanisms of action, combinations may be tried and the treatment of dystonia should be tailored to the individual patient. Drugs used to treat dystonia or related tremor disorders include but are not limited to anticholinergic agents, benzodiazepines, baclofen, dopamine and botulinum toxin. Anticholinergics include such drugs as Artane (trihexyphenidyl), Cogentin (benztropine), or Parsitan (ethopropazine) which block the acetylcholine. Typically, doses should be selected that do not, or only cause limited amounts of, confusion, drowsiness, hallucination, personality change, and memory difficulties, and peripheral side effects such as dry mouth, blurred vision, urinary retention, and constipation. Benzodiazepines, such as Valium (diazepam), Klonopin (clonazepam), and Ativan (lorazepam) block the Gaba-A receptor in the central nervous system and have been found useful for treating dystonia. The primary side effect is sedation, but others include depression, personality change, and drug addiction. Rapid discontinuation can result in a withdrawal syndrome. Some dystonia patients may tolerate very high doses without apparent adverse effects. Baclofen (Lioresal) stimulates the Gaba-B receptor. Intrathecal (spinal infusion) forms of Baclofen are also available for use in treatment. Some patients with primary dystonia respond to drugs which increases dopamine such as Sinemet (levodopa) or Parlodel (bromocriptine); however, many patients respond to agents which block or deplete dopamine, such as standard anti-psychotics like Clozaril (clozapine), Nitoman (tetrabenazine), or Reserpine.

Any of the preceding methods may be used to treat a neuronal disorder such as the disorders selected from the group consisting of a movement disorder, a neurodegenerative disease, a neurodevelopmental disorder and a neurophyschiatric disease. Particularly, any of the preceding methods may be used to treat a neuronal disorder such as dystonia, Parkinson's disease or Huntington's disease.

In one embodiment, any of the preceding methods comprise inhibiting expression of wild-type Dyt1 in Purkinje cells at least 40% as measured by a suitable assay.

In another embodiment, the expression construct of any of the preceding methods comprises a duplexed antisense compound comprising a polynucleotide sequence of 8 to 80 nucleotides in length targeted to a nucleic acid molecule encoding Dyt1 with at least one natural or modified nucleobase forming an overhang at a terminus of said sequence; and (b) the complementary sequence of said sequence (a) having optionally at least one natural or modified nucleobase forming an overhang at a terminus of said complementary sequence; wherein said sequences (a) and (b), when hybridized, have at least one single-stranded overhang and at least one of terminus of said hybridized duplex, and wherein said duplex when interacted with a nucleic acid molecule encoding said Dyt1 will inhibit expression of TorsinA in Purkinje cells.

The invention further provides methods wherein the polynucleotide sequences specifically hybridize to a sequence of said Dyt1 within at least 8 to 80 nucleotides extending 5′ of nucleic acid 645 of SEQ ID NO: 1, extending 5′ of nucleic acid 719 of SEQ ID NO: 1, extending 5′ of nucleic acid 793 of SEQ ID NO: 1, extending 5′ of nucleic acid 969 of SEQ ID NO: 1, extending 5′ of nucleic acid 1334, or extending 5′ of nucleic acid 1439 of SEQ ID NO: 1. The invention also provides for methods wherein the polynucleotide sequence specifically hybridizes with nucleic acids 625 to 645 of SEQ ID NO: 1, 686 to 719 of SEQ ID NO: 1, 772 to 793 of SEQ ID NO: 1, 931 to 969 of SEQ ID NO: 1, 1299 to 1334 of SEQ ID NO: 1 or 1419 to 1439 of SEQ ID NO: 1.

In a further embodiment, the invention provides for methods for treating dystonia or other motor-deficient disorders comprising inhibiting expression of a Dyt1 in Purkinje cells comprising: contacting a cell expressing a Dyt1 with a double stranded RNA comprising a sequence capable of hybridizing to Dyt1 mRNA corresponding to any one of the polynucleotide sequences of SEQ ID NOS: 3-14, in an amount sufficient to elicit RNA interference; and inhibiting expression of the Dyt1 gene in the Purkinje cell.

In particular, the invention provides for methods wherein the double stranded RNA is provided by introducing a short interfering RNA (siRNA) into the cell by a method selected from the group consisting of transfection, electroporation, and microinjection.

Alternatively, the invention provides for methods wherein the double stranded RNA is provided by introducing a short interfering RNA (siRNA) into the cell by an expression vector. The expression vector of these methods may comprise a Purkinje specific promoter operatively linked to said siRNA such as the Pcp2 promoter. The expression vector of these methods may also be a viral expression vector such as an adenoassociated viral vector.

The invention further provides for any of the preceding methods wherein said method provides an improved motor coordination in a mammal having a neuronal disease In addition, the invention also provide for an improved balance in mammals having a neuronal disease.

Other features and advantages of the invention will become apparent from the following detailed description. It should be understood, however, that the detailed description and the specific examples, while indicating preferred embodiments of the invention, are given by way of illustration only, because various changes and modifications within the spirit and scope of the invention will become apparent to those skilled in the art from this detailed description.

### BRIEF DESCRIPTION OF THE DRAWINGS

The following drawings form part of the present specification and are included to further illustrate aspects of the present invention. The invention may be better understood by reference to the drawings in combination with the detailed description of the specific embodiments presented herein.

FIG. 1. Generation of Dyt1 KO mice. FIG. 1A, The targeting vector used for the generation of Dyt1 KO mouse that would have exons 3 and 4 deleted. The sizes and locations of the restriction fragments for the identification of targeted clones are indicated. Black rectangles: exons; X: XbaI site; large black arrowhead: loxP sequence; large open arrowhead: FRT sequence; large open arrow: PGKNeo cassette for drug selection; small arrowheads: location and direction of PCR primers for genotyping. FIG. 1B, Southern analysis of transfected ES cell colonies performed to identify clones that homologously recombined the targeting construct; open arrowhead: wild-type clone, closed arrowhead: targeted clone. FIG. 1C, Gel electrophoresis picture of PCR products from the two homozygous knockout pups (Δ/Δ) found dead on the day of birth. The top gel showed that exons 3 and 4 were deleted from the mice. The bottom showed that loxP sequences were recombined and missing from both alleles of the KO mice. Small arrowheads: PCR primers used which correspond to primers in FIG. 1B.

FIG. 2. Generation of Dyt1 KD mice. FIG. 2A, Targeting construct and genomic structure of Dyt1 gene. Exon 5 in the targeting construct carried a GAG deletion, which was not homologously recombined into the modified allele of targeted ES cells due to a frequent recombination spot before the ΔGAG site. The sizes and locations of the restriction fragments for the identification of targeted clones are indicated. Black rectangles: exons; black arrowheads: loxP sites; open arrow: PGKNeoSTOP cassette; X: XbaI site; A, AhdI site. FIG. 2B, Southern analyses of the transfected ES cell colonies performed to identify clones that homologously recombined the targeting construct, open arrowhead: WT clone, closed arrowhead: targeted clone. FIG. 2C, Northern analysis of total RNA samples from brain tissues of WT and Dyt1 KD mice showed a reduction of torsinA mRNA in the Dyt1 KD brain. G3PDH quantity was used for loading control.

FIG. 3. Motor performance of 6 to 8 month-old Dyt1 KD mice on rotarod, beam-walking and pawprint tests. Male Dyt1 KD mice at 6-8 months of age displayed excessive slips on the beam-walking test and gait abnormality with severity. FIG. 3A, Dyt1 KD mice performed comparable to WT mice on the accelerated rotarod test. 1-6: trial number. FIG. 3B, Time of latency to crossing on the elevated beams in the beam-walking test was similar in Dyt1 KD mice as in WT mice. m Sq: medium square; m Rnd: medium round; s Sq: small square; s Rnd: small round. FIG. 3C, Male Dyt1 KD mice showed a significantly larger number of slips as they crossed the beam with an almost 200% increase in relative number of slips (WT mice=1 slip). FIG. 3D, The stride length of Dyt1 KD mice was normal in comparison to WT mice. FIG. 3E, Only male Dyt1 KD mice showed a significantly smaller hindpaw base than WT mice. FIG. 3D and FIG. 3E, R: right; L: left; Fore: forelimb; Hind: hindlimb; FIG. 3F: female; M: male. F, Dyt1 KD mice showed normal paw overlaps distances in comparison to WT mice; **p<0.01.

FIG. 4. Performance of Dyt1 KD mice on the open-field analysis. Male Dyt1 KD mice at 8-9 months of age exhibited hyperactivity and increased stereotypic activity in open-field analysis. FIG. 4A, Horizontal activity was significantly higher in male Dyt1 KD mice than WT controls. FIG. 4B, No difference was observed in vertical activity of Dyt1 KD versus WT mice. FIG. 4C and FIG. 4D, Stereotypic count and number were both higher in male Dyt1 KD mice than WT controls.*p<0.05.

FIG. 5. Generation of AGAG knockin mice. FIG. 5A. Targeting construct and the genomic organization of Dyt1 gene. Exon 5 in the targeting construct carried a GAG deletion. The PGKNeoSTOP cassette was inserted into intron 4. The sizes and locations of the restriction fragments for the identification of targeted clones are indicated. Black rectangles: exons; black arrowheads: loxP sites; open arrow: PGKNeoSTOP cassette; X: XbaI site; A, AhdI site. FIG. 5B. Representative southern blot of the transfected ES cell colonies. WT: wild-type locus, MT: mutant locus; closed arrowhead targeted clone; open arrowhead: untargeted clone. FIG. 5C. cDNA sequence of torsinA transcript in ΔGAG Dyt1 heterozygous germline transmitted pup. Two different transcripts are present, one with the GAG (WT allele) and one without GAG (mutated allele).

FIG. 6. Male AGAG Dyt1 mice at 6-8 months of age displayed excessive slips on the beam-walking test and gait abnormality with severity. FIG. 6A. ΔGAG Dyt1 mice performed comparable to WT mice on the accelerated rotarod test. 1-6: trial number. FIG. 6B. Time of latency to crossing on the elevated beams in the beam-walking test was similar in ΔGAG Dyt1 mice as in WT mice. m Sq: medium square; m Rnd: medium round; s Sq: small square; s Rnd: small round. FIG. 6C. Male ΔGAG Dyt1 mice showed a significantly larger number of slips as they crossed the beam with an almost 300% increase in relative number of slips (WT mice=1 slip; p=0.013).

FIG. 7. Male mice have an abnormality gait pattern of paw overlap. FIG. 7A and FIG. 7B. Pawprints of WT and ΔGAG Dyt1 respectively do not exhibit obvious debilitating gait abnormalities. FIG. 7C and FIG. 7D. Measurements of the stride and base lengths, respectively, of fore and hindpaws showed no significant differences between ΔGAG Dyt1 and WT mice. FIG. 7E. The overlap measurement showed a difference between ΔGAG Dyt1 and WT, but only between the males (p=0.021). purple, hindlimb; orange: forelimb;

FIG. 8. ΔGAG Dyt1 mice are hyperactive in the open-field analysis. FIG. 8A. Horizontal activity was significantly higher in male Dyt1 KD mice than WT controls (p=0.034). FIG. 8B. Vertical activity between ΔGAG Dyt1 and control mice did not differ (p=0.68). FIG. 8C. Total distance traveled showed was significant higher in ΔGAG Dyt1 than WT male mice (p=0.010). D. Male ΔGAG Dyt1 mice traveled more distance in the marginal area of the open-field apparatus than did WT mice

FIG. 9. In situ hybridization using riboprobes specific for RGS9L in parasagittal planes (B) and coronal planes through rat striatum (str; C, D). Note that RGS9L mRNA is most dense in striatum and olfactory tubercle (ot); ob, olfactory bulb. Scale bar (shown in B): 2.3 mm; C, D, 1.4 mm. modified from [1]

FIG. 10. β-galactosidase staining of sagittal brain section from P60 mice that were positive both for RGS9L-cre knock-in and Rosa reporter genes. The staining was restricted to striatum/nucleus-accumbens.

FIG. 11. Compared to control mice, sKO and male cKO mice showed significantly more slips, while pKO mice showed significantly less slips during beam walking tests. *, p<0.05.

FIG. 12. At about 4 months of age, cKO mice (A, D) showed hyperactivity in open field tests while sKO (B, E) and pKO (C, F) mice exhibited normal level of exploratory activity. *, p<0.05.

FIG. 13. At about 7 months of age, cKO mice (A, D) continued to show hyperactivity in open field tests while sKO (B, E) and pKO (C, F) mice exhibited normal level of exploratory activity. *, p<0.05. Note LD mice started to show hyperactivity similar to Dyt1 KD and ΔGAG knock-in mice.

FIG. 14. Silencing of DYT1 gene by U6shTAcom. FIG. 14A: Western detection (WB) of proteins indicated on the right. Cos7 cells were cotransfected with TAwtGFP, HA-TAmut and TOPO-shTAmis (missense, not targeting any known sequence) or TOPO-shTAcom (targeting a sequence shared by human and mouse TA). While the missense did not have effects on TA levels, shTAcom silenced both wt and mutant TA expression. shTA expression was driven by a U6 promoter. FIG. 14B: Quantification of WB signal in 3 independent experiments.

FIG. 15. Alignment demonstrating the homology between human and mouse torsinA cDNA sequences. Nucleotides 525-1970 of the human torsinA gene (Genbank accession no. AF007871; SEQ ID NO: 1) are displayed as nucleotides 1-1446 (upper sequence). Nucleotides 26-1355 of the mouse torsinA gene (Genbank accession no. NM_{—}144884; SEQ ID NO: 15) are displayed as nucleotides 1-1339 (lower sequence). The underlined sequences are those which have stretches of more than 20 identical nucleotides.

FIG. 16. Excerpt of FIG. 2 from Gonzalez-Alegre et al., (Ann Neurol 2003; 53:781-787 which describes the design and targeted sequences of small interfering RNAs (siRNAs). Shown are the relative positions and targeted mRNA sequences for each primer used in this study. Mis-siRNA (negative control) does not target TA; com-siRNA targets a sequence present in wild-type and mutant TA; wt-siRNA targets only wild-type TA; and three mutant-specific siRNAs (mutA, B, C) preferentially target mutant TA. The pair of GAG codons near the C terminus of wild-type mRNA are shown in underlined gray and black, with one codon deleted in mutant mRNA.

### DETAILED DESCRIPTION OF THE PREFERRED EMBODIMENTS OF THE INVENTION

Movement disorders constitute a major group of human neurologic diseases in which aberrant neurotransmission in the basal ganglia is associated with uncontrollable body movements, such as chorea in Huntington disease, tremor and rigidity in Parkinson disease, and twisting contraction in torsion dystonia. The clinical manifestations of dystonia show wide variations in age and site of onset, as well as body regions involved. Early onset, generalized dystonia is the most disabling form of primary dystonia in which symptoms usually begin in an arm or leg at around 12 yrs (range 4-44 years) and spread to involve other limbs within about 5 years (Bressman, S. B. et al., Annal Neurol 36.771-777 (1994b); Greene, P. et al., Mov Disord 10: 143-152 (1995)). The clinical spectrum of early onset dystonia is similar in all ethnic populations, with highest prevalence in the Ashkenazi Jewish (termed here AJ) population (Zeman, W., & Dyken, P., Psychiatr Neurol Neurochir 10:77-121 (1967); Korczyn, A. D. et al., Ann Neurol 8:387-391 (1980); Eldridge, R., Neurology 20:1-78 (1970)), due to a founder mutation (Ozelius, L. et al., Am. J Hum. Genet. 50:619-628 (1992); Risch, N. J. et al., Nature Genetics 9:152-159 (1995)). While the genetic mutations responsible for dystonia has been identified, there remains a need to produce effective treatments for this debilitating class of disorders.

As explained above, dystonia is believed to result from a loss-of-function mutation in Dyt1. As such, it would be expected that it would be desirable to provide a therapeutic intervention by increasing or augmenting the activity and/or expression of the wild-type product of the Dyt1 gene. Surprisingly, however, in the present invention, it is found that a selective inactivation of the wild-type Dyt1 in Purkinje cells of mammals exhibiting dystonia results in an improved balance and motor coordination in said mammals. Thus, rather than increasing the activity and/or expression of the Dyt1, the present invention provides methods and compositions for decreasing the activity and/or expression of Dyt1 in order to achieve a therapeutic outcome.

The data described in the examples herein below show the successful generation of a knockin of Dyt1 AGAG, knockdown of wild-type torsinA, knockout of torsinA, and three tissue-specific knockout mouse lines of Dyt1. Motor coordination and balance analyses of Dyt1 knockdown (KD), Dyt1ΔGAG knockin (KI), and Dyt1 heterozygous knockout mice (LD) mice are described. Dyt1 KD, KI, and LD mice showed similar motor deficits suggesting Dyt1ΔGAG mutation is a loss-of-function mutation. Finally, the data show that cerebral cortex-specific or striatum-specific, but not Purkinje cell-specific, inactivation of Dyt1 gene in mice could produce motor deficits that were present in KI mice. Surprisingly, when Dyt1 gene was inactivated in Purkinje cells, the mutant mice showed significantly improved motor coordination and balance.

Cerebellar circuits especially Purkinje cells are important in movement and posture control, and therefore these cells are a good target for improving the symptoms of dystonia. Genetically the dystonic rat (dt rat) model described herein is a spontaneous mutation that results in severe dystonic movement posture and early postnatal death. Cerebellectomy eliminates the motor syndrome of the dt rat and rescues the mutant rat from juvenile lethality [43]. Electronic lesions of the dorsal portions of the lateral vestibular nuclei (dLV), which receives input from Purkinje cells, are associated with the greatest motor improvement in dt rats [44]. The abnormal output from dLV to spinal cord in dt rat is likely originated from its Purkinje cells, since abnormal spontaneous and harmaline-stimulated Purkinje cell activity has been detected in dt rats [45]. Specifically, dt rat fired less simple and complex spikes before and after harmaline treatment and less simple spikes only after harmaline treatment. Similarly, pharmacological disruption of cerebellar signaling induces dystonia in mice [46]. Cerebellectomy or vermectomy improves motor performance in Weaver mutat mice, an animal model for cerebellar ataxia [47,48]. Tottering mouse, which has a recessive mutation of the calcium channel gene and showed ataxia and paroxysmal dystonia, showed no dystonia phenotype when introduced into selective Purkinje cell degeneration (pcd) background [49]. Brain imaging of dystonia patients also showed abnormal activity in cerebellum [50]. DYT1ΔGAG carrier in human show significant increased metabolism in cerebellar hemispheres while no changes were observed in DYT6 carriers [51 ]. Taken together, abnormal function of cerebellar circuits is likely involved in the pathogenesis of DYT1 dystonia.

The improvement effect of Dyt1 inactivation in Purkinje cells provides a novel therapeutic strategy for DYTI dystonia. In the present invention Purkinje cell-specific viral expression of shRNA against wild type Dyt1 mRNA is contemplated to improve motor performance in dystonia. More specifically, it is proposed to use Adeno-Associated Virus (AAV) to deliver therapeutically effective amounts of shRNA against wild type Dyt1 to Purkinje cells under the control of a Purkinje cell-specific promoter.

The invention specifically contemplates the use of RNAi technology, to turn off the Dyt1 gene they want to study. RNAi technology is faster than conventional knockout approach in mice and provides a cheaper alternative than the ES cell-based gene targeting. Originally, synthetic RNAs were used. Recently, with the development of RNA polymerase III-mediated transcription, shRNA (short hairpin RNA) can be produced in vivo using virus vectors. Coupled with cre-loxP technology, conditional expression of shRNA has been achieved in mice [52,53] and may readily be modified for the present applications.

Wild-type AAV is a non-pathogenic human parvovirus. It requires helper functions from other viruses to replicate and can function as a recombinant vector with all the viral genes removed, it is extremely safe to use and has been the choice for a few ground breaking clinical trials and animal models of neurological disorders [54]. Trials are underway for Parkinson's disease and other neurological disorders using AAV vectors (detailed and updated information can be obtained at http://www.gemcris.od.nih.gov/Contents/GC_HOME.asp). Recently, AAV-based shRNA has been used to suppress the polyglutamine-induced neurodegeneration in a mouse model of spinocerebellar ataxia type 1 (SCA1) [55]. SCA1 mice contain a transgenic human disease allele (ataxin-1-Q82) controlled by Purkinje cell-specific PCP-2 promoter. The shRNA against human SCA1 sequence was used to successfully downregulate the expression of transgene without any effect on endogenous mouse Sca1 gene. Upon intracerebellar injection of the AAV virus that expresses the shRNA against SCA1 sequence, improvement in motor coordination, restored cerebellar morphology, and resolved characteristic ataxin-1 inclusions were achieved. The same group also succeeded in achieving improvement in motor performance and neuropathological phenotypes in a Huntington's disease mouse model using similar strategies [56]. These pioneering experiments demonstrated the potential to use RNAi to treat human movement disorders. In the present invention, similar methods are used in order selectively inactivate Dyt1 in mammals and to achieve inhibit of the wild type gene expression. The shRNA molecules of the invention are complementary to both mouse and human torsinA gene. The present invention demonstrates the effectiveness of these molecules against the human DYT1 gene in cultured human cells and in mammals. Therefore, preferably, the treatment methods of the invention are carried out for the treatment of humans, however, the methods also may be performed in other mammals, including but not limited to other primates, farm animals including cows, sheep, pigs, horses and goats, companion animals such as dogs and cats, exotic and/or zoo animals, laboratory animals including mice rats, rabbits, guinea pigs and hamsters.

Thus, the following specification provides details for performing Purkinje cell-specific viral expression of shRNA against wild type Dyt1 mRNA in order to improve motor performance. The invention is directed towards Purkinje cell-specific Dyt1 inactivation in Dyt1ΔGAG knock-in mice to improve their motor performance. AAV vector constructs are used to achieve Purkinje cell-specific silencing of the endogenous Dyt1 gene. The AAV vector will be packaged and virus particles will be injected intracerebellarly to achieve Purkinje cell-specific silencing of the Dyt1 gene in Dyt1ΔGAG knock-in mice. The motor coordination and balance will be tested in these mice to determine whether there is any improvement over Dyt1ΔGAG knock-in mice treated with a control AAV virus that has shRNA against a lacZ gene.

The term “expression” with respect to a gene sequence refers to transcription of the gene and, as appropriate, translation of the resulting mRNA transcript to a protein. Thus, as will be clear from the context, expression of a protein coding sequence results from transcription and translation of the coding sequence. A method that decreases the expression of a gene may do so in a variety of ways (none of which are mutually exclusive), including, for example, by inhibiting transcription of the gene, decreasing the stability of the mRNA and decreasing translation of the mRNA. While not wishing to be bound to a particular mechanism, it is generally thought that siRNA techniques decrease gene expression by stimulating the degradation of targeted mRNA species.

By “silencing” a target gene herein is meant decreasing or attenuating the expression of the target gene.

A. RNAi Technology

RNA interference(RNAi) , also known as small interfering RNA (siRNA), is a particularly useful technique for reducing or disrupting the expression of a gene is. “RNA interference” was first used by researchers studying C. elegans and describes a technique by which post-transcriptional gene silencing (PTGS) is induced by the direct introduction of double stranded RNA (dsRNA: a mixture of both sense and antisense strands). Injection of dsRNA into C. elegans resulted in much more efficient silencing than injection of either the sense or the antisense strands alone (Fire et al., Nature 391:806-811, 1998). Just a few molecules of dsRNA per cell is sufficient to completely silence the expression of the homologous gene. Furthermore, injection of dsRNA caused gene silencing in the first generation offspring of the C. elegans indicating that the gene silencing is inheritable (Fire et al., Nature 391:806-811, 1998). Current models of PTGS indicate that short stretches of interfering dsRNAs (21-23 nucleotides; siRNA also known as “guide RNAS”) mediate PTGS. siRNAs are apparently produced by cleavage of dsRNA introduced directly or via a transgene or virus. In exemplary embodiments of the present invention, the dsRNAs are introduced under the control of a Purkinje-cell specific promoter in an adenoassociated virus vector. Of course, it should be understood that the dsRNAs may be delivered directly as small molecules or as transgenes. Moreover, while the exemplary promoter used herein is PcP, the siRNAs may be under the expression control of any promoter that is specifically expressed in Purkinje cells. Furthermore, while the exemplary embodiments employ adenoassociated virus as a vector, any viral vectors may be used. In particular, it is contemplated that lentiviral vectors may be particularly useful as these vectors will allow for larger amounts of nucleic acid to be transported. In this regard, it is now widely recognized that DNA may be introduced into a cell using a variety of viral vectors. In such embodiments, expression constructs comprising viral vectors containing the genes of interest may be adenoviral (see for example, U.S. Pat. No. 5,824,544; U.S. Pat. No. 5,707,618; U.S. Pat. No. 5,693,509; U.S. Pat. No. 5,670,488; U.S. Pat. No. 5,585,362; each incorporated herein by reference), retroviral (see for example, U.S. Pat. No. 5,888,502; U.S. Pat. No. 5,830,725; U.S. Pat. No. 5,770,414; U.S. Pat. No. 5,686,278; U.S. Pat. No. 4,861,719 each incorporated herein by reference), adeno-associated viral (see for example, U.S. Pat. No. 5,474,935; U.S. Pat. No. 5,139,941; U.S. Pat. No. 5,622,856; U.S. Pat. No. 5,658,776; U.S. Pat. No. 5,773,289; U.S. Pat. No. 5,789,390; U.S. Pat. No. 5,834,441; U.S. Pat. No. 5,863,541; U.S. Pat. No. 5,851,521; U.S. Pat. No. 5,252,479 each incorporated herein by reference), an adenoviral-adenoassociated viral hybrid (see for example, U.S. Pat. No. 5,856,152 incorporated herein by reference) or a vaccinia viral or a herpesviral (see for example, U.S. Pat. No. 5,879,934; U.S. Pat. No. 5,849,571; U.S. Pat. No. 5,830,727; U.S. Pat. No. 5,661,033; U.S. Pat. No. 5,328,688 each incorporated herein by reference) vector.

Several non-viral methods for the transfer of nucleic acid constructs into cultured mammalian cells are contemplated by the present invention. These include calcium phosphate precipitation (Graham and Van Der Eb, Virology, 52:456-467, 1973; Chen and Okayama, Mol. Cell Biol., 7:2745-2752, 1987; Rippe et al., Mol. Cell Biol., 10:689-695, 1990) DEAE-dextran (Gopal, Mol. Cell Biol., 5:1188-1190, 1985), electroporation (Tur-Kaspa et al., Mol. Cell Biol., 6:716-718, 1986; Potter et al., Proc. Nat. Acad. Sci. USA, 81:7161-7165, 1984), direct microinjection (Harland and Weintraub, J. Cell Biol., 101:1094-1099, 1985.), DNA-loaded liposomes (Nicolau and Sene, Biochim. Biophys. Acta, 721:185-190, 1982; Fraley et al., Proc. Natl. Acad. Sci. USA, 76:3348-3352, 1979; Felgner, Sci Am. 276(6):102 6, 1997; Felgner, Hum Gene Ther. 7(15):1791 3, 1996), cell sonication (Fechheimer et al., Proc. Natl. Acad. Sci. USA, 84:8463-8467, 1987), gene bombardment using high velocity microprojectiles (Yang et al., Proc. Natl. Acad. Sci USA, 87:9568-9572, 1990), and receptor-mediated transfection (Wu and Wu, J. Biol. Chem., 262:4429-4432, 1987; Wu and Wu, Biochemistry, 27:887-892, 1988; Wu and Wu, Adv. Drug Delivery Rev., 12:159-167, 1993).

Regardless of the vector and promoter used to effect Purkinje-specific delivery of the siRNA molecules, the siRNAs may be amplified by an RNA-dependent RNA polymerase (RdRP) and are incorporated into the RNA-induced silencing complex (RISC), guiding the complex to the homologous endogenous mRNA, where the complex cleaves the transcript. Thus, siRNAs are nucleotides of a short length (typically 18-25 bases, preferably 19-23 bases in length) which incorporate into an RNA-induced silencing complex in order to guide the complex to homologous endogenous mRNA for cleavage and degradation of the transcript.

While most of the initial studies were performed in C. elegans, RNAi has gained significant prominence as a technique that may be used in mammalian cells. It is contemplated that RNAi, or gene silencing, will be particularly useful in the disruption of Dyt1 expression, and this may be achieved in a tissue-specific manner in Purkinje cells. By placing a gene fragment encoding the desired dsRNA behind an inducible or tissue-specific promoter, it will be possible to inactivate genes at a particular location within an organism or during a particular stage of development. In this regard, in recent studies an AAV-based siRNA (called a silencing hairpin RNA shRNA) has been used to suppress the polyglutamine-induced neurodegeneration in a mouse model of spinocerebellar ataxia type 1 (SCA1) [55]. SCA1 mice contain a transgenic human disease allele (ataxin-1-Q82) controlled by Purkinje cell-specific PCP-2 promoter. The PCP-2 promoter is described in detail in Oberdick et al., Neuron, Vol. 10, 1007-1018, June, 1993, (incorporated herein by reference). The shRNA against human SCA1 sequence was used to successfully down-regulate the expression of transgene without any effect on endogenous mouse Sca1 gene. Upon intracerebellar injection of the AAV virus that expresses the shRNA against SCA1 sequence, improvement in motor coordination, restored cerebellar morphology, and resolved characteristic ataxin-1 inclusions were achieved. The same group also succeeded in achieving improvement in motor performance and neuropathological phenotypes in a Huntington's disease mouse model using similar strategies [56]. These prior studies are particularly instructive as they show that neuronal cell-specific expression of a siRNA molecule may be readily achieved using techniques in the art.

Variations on RNA interference (RNAi) technology is revolutionizing many approaches to experimental biology, complementing traditional genetic technologies, mimicking the effects of mutations in both cell cultures and in living animals. (McManus & Sharp, Nat. Rev. Genet. 3, 737-747 (2002)). RNAi has been used to elicit gene-specific silencing in cultured mammalian cells using 21-nucleotide siRNA duplexes (Elbashir et al., Nature, 411:494-498, 2001; Fire et al., Nature 391, 199-213 (1998), Hannon, G. J., Nature 418, 244-251 (2002))). In the same cultured cell systems, transfection of longer stretches of dsRNA yielded considerable nonspecific silencing. Thus, RNAi has been demonstrated to be a feasible technique for use in mammalian cells and could be used for assessing gene function in cultured cells and mammalian systems, as well as for development of gene-specific therapeutics. In particularly preferred embodiments, the siRNA molecule is between 20 and 25 oligonucleotides in length and is derived from the sequence of SEQ ID NO: 1. Particularly preferred siRNA molecules are 21-23 bases in length.

The siRNA molecules of the present invention can be obtained using a number of techniques known to those of skill in the art. For example, the siRNA can be chemically synthesized or recombinantly produced using methods known in the art. For example, short sense and antisense RNA, DNA or XNA oligomers can be synthesized and annealed to form double-stranded structures with 2-nucleotide overhangs at each end (Caplen, et al. (2001) Proc Natl Acad Sci USA, 98:9742-9747; Elbashir, et al. (2001) EMBO J, 20:6877-88). These double-stranded siRNA structures can then be introduced into cells, either by passive uptake or a delivery system of choice.

The siRNA molecules can be purified using a number of techniques known to those of skill in the art. For example, gel electrophoresis can be used to purify siRNAs. Alternatively, non-denaturing methods, such as non-denaturing column chromatography, can be used to purify the siRNA. In addition, chromatography (e.g., size exclusion chromatography), glycerol gradient centrifugation, affinity purification with antibody can be used to purify siRNAs.

In certain preferred embodiments, at least one strand of the siRNA molecules has a 3′ overhang from about 1 to about 6 nucleotides in length, though may be from 2 to 4 nucleotides in length. More preferably, the 3′ overhangs are 1-3 nucleotides in length. In certain embodiments, one strand having a 3′ overhang and the other strand being blunt-ended or also having an overhang. The length of the overhangs may be the same or different for each strand. In order to further enhance the stability of the siRNA, the 3′ overhangs can be stabilized against degradation. In one embodiment, the RNA antisense strand is stabilized by including purine nucleotides, such as adenosine or guanosine nucleotides. Alternatively, substitution of pyrimidine nucleotides by modified analogues, e.g., substitution of uridine nucleotide 3′ overhangs by 2′-deoxythyinidine is tolerated and does not affect the efficiency of RNAi. The absence of a 2′ hydroxyl significantly enhances the nuclease resistance of the overhang in tissue culture medium and may be beneficial in vivo.

In certain embodiments, an RNAi construct is in the form of a hairpin structure. The hairpin can be synthesized exogenously or can be formed by transcribing from RNA polymerase III promoters in vivo. Examples of making and using such hairpin RNAs for gene silencing in mammalian cells are described in, for example, Paddison et al., Genes Dev, 2002, 16:948-58; McCaffrey et al., Nature, 2002, 418:38-9; McManus et al., RNA, 2002, 8:842-50; Yu et al., Proc Natl Acad Sci USA, 2002, 99:6047-52). Preferably, such hairpin RNAs are engineered in cells or in an animal to ensure continuous and stable suppression of a desired gene. It is known in the art that siRNAs can be produced by processing a hairpin RNA in the cell. A hairpin may be chemically synthesized such that a sense strand comprises RNA, DNA or XNA, while the antisense strand comprises RNA. In such an embodiment, the single strand portion connecting the sense and antisense portions should be designed so as to be cleavable by nucleases in vivo, and any duplex portion should be susceptible to processing by nucleases such as Dicer.

Commercial providers such as Ambion Inc. (Austin, Tex.), Darmacon Inc. (Lafayette, Co.), InvivoGen (San Diego, Calif.), and Molecula Research Laboratories, LLC (Herndon, Va.) generate custom siRNA molecules. In addition, commercial kits are available to produce custom siRNA molecules, such as SILENCER™ siRNA Construction Kit (Ambion Inc., Austin, Tex.) or psiRNA System (InvivoGen, San Diego, Calif.). These siRNA molecules may be introduced into cells through transient transfection or by introduction of expression vectors that continually express the siRNA in transient or stably transfected mammalian cells. Transfection may be accomplished by well known methods including methods such as infection, calcium chloride, electroporation, microinjection, lipofection or the DEAE-dextran method or other known techniques. These techniques are well known to those of skill in the art.

In particularly preferred embodiments, the shRNA molecules of the present invention are designed to target the nucleic acid that encodes TorsinA. The nucleic acid that encodes TorsinA is depicted in SEQ ID NO:1 and the protein encoded by said nucleic acid is depicted in SEQ ID NO:2. U.S. Patent Publication No. 20010029015 is incorporated herein by reference in its entirety and provides a specific teaching of detecting mutations and polymorphisms in the torsin gene, torsin-related genes, methods of detecting neuronal diseases mediated by these mutations and polymorphisms and nucleic acids used in these methods. U.S. Patent Publication No. 20030235823 also is expressly incorporated by reference and provides a teaching of torsin-encoding genes, torsin proteins, and methods of using the same to treat protein-aggregation.

The Dyt1 gene shown in SEQ ID NO:1 is 2597 nucleotides in length with the open reading frame beginning at nucleotide 568 and ending at nucleotide 1563. The shRNA molecules of the invention may be designed from any point along that 2597 length of the gene. In particular, the shRNA molecules should be designed to target the region between 568 and 1563 of SEQ ID NO:1. The specific sequences of the shRNA molecules may be prepared by designing 21-23 nucleotide stretches from the entire region. Such stretches may be overlapping. For example, a first shRNA molecule may be one that targets nucleotides 568 to 589 of SEQ ID NO:1, a second shRNA molecules may be one that targets nucleotides 569 to 590 of SEQ ID NO:1, in like manner other exemplary shRNAs target 570 to 591; 571 to 592; 572 to 593; 573 to 594, 574 to 595; 575 to 596; 576 to 597; 577 to 598; 578 to 599; 579 to 600; 580 to 601, 931 to 969 etc. all the way through to residue 1563. The skilled person should prepare a complete set of shRNA by walking along the gene of SEQ ID NO:1 all the way from 568 to 1563 and test these molecules using the methods described herein using the exemplary molecule discussed in Example 2. Similar sets of shRNA molecules can be prepared that are 22 nucleotides in length e.g., 568 to 590; 569 to 591; 570 to 592 etc., and 23 nucleotides in length e.g., 568 to 591; 569 to 592; 570 to 593 etc. Such complete sets of shRNA test molecules can be created using routine techniques. Once molecules that have a positive effect when expressed in Purkinje cells are identified from these sets using methods such as those described in Example 2, those molecules can then be prepared as pharmaceutical compositions for the treatment of dystonia.

Treatment of Dystonia and Related Disorders

Once the shRNA molecules are identified and tested for efficacy in test animals as discussed above, the present invention further contemplates methods of treating, reducing, arresting, alleviating, ameliorating, or preventing symptoms of dystonia. Such methods may involve administration of the shRNA molecules alone or a combination of the shRNA-based therapy with other compounds or drugs that are used for reducing, arresting, alleviating, ameliorating, or preventing symptoms of motor-deficient disorders including dystonia and dystonia-related disorders such as Huntington's disease and Parkinson's disease.

The disorders to be treated may include neurodegenerative diseases or disorders, primary dystonia (preferably, generalized dystonia and torsion dystonia). Dystonia-related diseases include dystonic tremor, Parkinson's disease, tremor, fibromyalgia, Hallervorden-Spatz syndrome, congenital torticollis, and Wilson's disease.

Gene therapy methods can be used to transfer the shRNA molecules that target the torsin coding sequence of the invention to a patient (Chattedee and Wong, 1996, Curr. Top. Microbiol. Immunol. 218:61-73; Zhang, 1996, J. Mol. Med. 74:191-204; Schmidt-Wolf and Schmidt-Wolf, 1995, J. Hematotherapy. 4:551-561; Shaughnessy, et al., 1996, Seminars in Oncology. 23:159-171; Dunbar, 1996,Annu. Rev. Med. 47:11-20). A “patient” or “subject” to be treated by a disclosed method can mean either a human or non-human animal.

The preferred vectors used to achieve the gene therapy are adenoassociated viral vectors. However, examples of other vectors that may be used in gene therapy include, but are not limited to, defective retroviral, adenoviral, or other viral vectors (Mulligan, R. C., 1993, Science. 260:926-932). The means by which the vector carrying the gene can be introduced into the cell include but is not limited to, microinjection, electroporation, transduction, or transfection using DEAE-Dextran, lipofection, calcium phosphate or other procedures known to one skilled in the art (Sambrook, J., Fritsch, E. F., and Maniatis, T., 1989, In: Molecular Cloning. A Laboratory Manual., Cold Spring Harbor Laboratory Press, Cold Spring Harbor).

The treatment methods of the invention particularly contemplate administering shRNA molecules to an animal (preferably, a mammal (specifically, a human)) in an amount sufficient to effect a silencing of the Dyt1 gene in the Purkinje cells of the animal.

One skilled in the art will appreciate that the amounts to be administered for any particular treatment protocol can readily be determined. The dosage should not be so large as to cause adverse side effects, such as unwanted cross-reactions, anaphylactic reactions, and the like. Generally, the dosage will vary with the age, condition, sex and extent of disease in the patient, counter indications, if any, and other such variables, to be adjusted by the individual physician. Dosage can vary from 0.001 mg/kg to 50 mg/kg of Dyt1-targeting shRNA, in one or more administrations daily, for one or several days. The shRNA molecules can be administered parenterally by injection or by gradual perfusion over time. It can be administered intravenously, intraperitoneally, intramuscularly, or subcutaneously. Preferably the administration is via intracerebellar injection or by use of an intrathecal catheter.

The dosage forms of the injectable preparations (solutions, suspensions, emulsions, solids to be dissolved when used, etc.), tablets, capsules, granules, powders, liquids, liposome inclusions, ointments, gels, external powders, sprays, inhalating powders, eye drops, eye ointments, suppositories, pessaries, and the like can be used appropriately depending on the administration method, and the peptide of the present invention can be accordingly formulated. Pharmaceutical formulations are generally known in the art, and are described, for example, in Chapter 25.2 of Comprehensive Medicinal Chemistry, Volume 5, Editor Hansch et al, Pergamon Press 1990 and Remington's Pharmaceutical Science, 16th ed., Eds.: Osol, A., Ed., Mack, Easton Pa. (1980).

The effectiveness of the therapy may readily be monitored using any of the diagnostic tests that are used to monitor motor skills in individuals suffering from dystonia. Such diagnostic tests may include the physical and neurological examination of the patient before and after the therapy. Such measurements are determined before the therapy to determine the baseline characteristics of the disorder being treated in the specific patient and the measurements are then taken again over the period of the therapy to determine the effectiveness thereof. Any alleviation or amelioration of the symptoms of the disorder will be indicative of the effectiveness of the therapy. For example, an dystonia is primarily characterized by an involuntary sustained twisting or cramping posture. Any alleviation of such symptoms in response to the methods of treatment of the invention will be indicative that the shRNA-based therapy is effective. Any alleviation of the symptoms of Parkinson's disease including the tremor, disorientation and the like will also be indicative of the therapeutic efficacy of the treatment methods of the invention.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

In order to study the pathophysiology of the DYTI dystonia, reverse genetic approach based on gene targeting/ES cell technologies was used to generate several lines of Dyt1 mutant mice. The generation and analysis of each line is described below. General gene targeting and ES cell technologies for preparing transgenic animals from such cell lines are well-established and well-known to those of skill in the art. For example, those skilled in the art are referred to U.S. Patent Publication No. 20010029015 which specifically provides a teaching of generation of torsin-related transgenic animals.

### Example 1

### Demonstration that Dyt1 is Required for Early Mouse Development: Perinatal Lethality of Dyt1 Knockout Mice

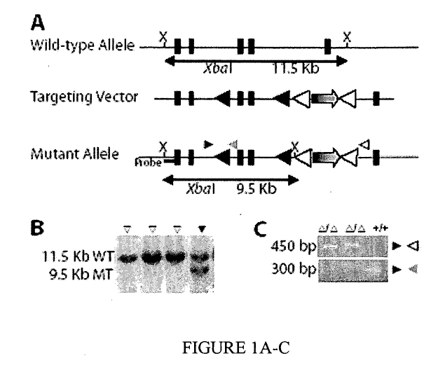

The Dyt1 gene of 129/Sv origin was first isolated from a Bacterial Artificial Chromosome Mouse I library, mapped, subcloned and sequenced. To study the in vivo function of torsinA protein, a knockout allele of Dyt1 gene was generated. Dyt1 knockout mice were generated from a line of Dyt1 loxP mouse that have exons 3 and 4 flanked by two loxP sites. The construct contained a loxP sequence in introns 2 and 4 with a PGK-neomycin cassette flanked by FRT sequences in intron 4 (FIG. 1A). Of the 121 transfected ES cell colonies screened, two were found to have undergone homologous recombination and correctly targeted the Dyt1 gene (FIG. 1B). These two clones were expanded and injected into C57BL/6 blastocysts to obtain chimeric mice. When germline transmitted pups were mated with CMV-cre mice [57], Cre caused the removal of exons 3 and 4 of Dyt1, which was verified by PCR (FIG. 1C). Interbreeding of heterozygous pups containing a Dyt1 knockout allele with the two missing exons yielded litters containing the WT and heterozygous pups, but no live homozygous Dyt1 knockout pups were found after postnatal day 1. Among the 46 live pups genotyped, 13 were wild-type and 33 were heterozygous Dyt1 KO (χ²=13.38, p≦0.01). Two additional dead homozygous Dyt1 KO pups were recovered on the day of birth. These mice did not have full milk sacs in the abdomen, which suggests they may possess a possible feeding deficit. The lethality of Dyt1 KO mice suggests torsinA is essential for normal development.

### Example 2

### Motor Deficits and Hyperactivity in Dyt1 KD Mice

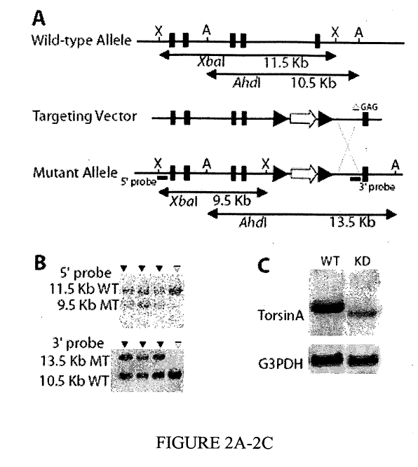

The targeting construct used to generate Dyt1 knockdown (KD) mice had a Dyt1 gene that carried the AGAG mutation (FIG. 2A). This construct was originally made to generate a knockin mouse line. Twenty eight of 73 clones screened had homologously recombined the targeting construct as determined by Southern blot analysis on both sides (FIG. 2B). When the 28 clones were further screened for the presence of the trinucleotide deletion, only three were found to carry the deletion. A highly efficient recombination site must have existed within intron 4 downstream of the PGKneoSTOP cassette and before the ΔGAG site in exon 5. As a result, all DNA sequences downstream of that recombinatorial site including the mutated exon 5 were not incorporated into the genomic DNA. Six of the 25 clones containing the PGKneoSTOP cassette and a wild-type Dyt1 sequence were expanded and injected to produce chimeric mice. The three clones that contained ΔGAG were used to generate a knock-in ΔGAG line as described below in Example 3.

The heterozygotes, (Dyt1 STOP +/−) that did not carry the ΔGAG mutation were interbred. If the termination of Dyt1 was efficiently blocked by the poly(A) tail and the STOP sequence, homozygous Dyt1 STOP−/− mice would resemble Dyt1Δ/Δ and no surviving pups of the Dyt1 STOP−/− genotype are expected to be seen. However, when Dyt1 STOP+/− mice were interbred, WT, heterozygous, and homozygous mice were generated at the expected Mendelian ratio of 1+/+:2+/−: 1−/−. To determine the effectiveness of the poly(A) tail and STOP sequence in terminating transcription, Dyt1 expression was measured. Northern blot analysis using a probe specific for exon 5 showed that approximately 45% of normal Dyt1 mRNA was present (n=3 each, FIG. 2C) in the Dyt1 STOP −/− mice. No additional RNA band was detected when hybridized with a probe covering exons 1 to 4 (data not shown), suggesting the absence of additional hybrid RNAs that could potentially produce hybrid proteins possessing just the N-terminal portion of torsinA. Dyt1 STOP −/− mice were then named Dyt1 KD mice. Dyt1 KD mice did not appear to have any visible developmental deficits.

Since Dyt1 KO mice were lethal and Dyt1 KD mice appeared to survive into adulthood without gross developmental abnormalities, the inventors sought to determine the survivability of an animal that contained about half of the torsinA that is found in the KD mice. Heterozygous KO animals (Dyt1 +/Δ) were bred with heterozygous KD (Dyt1 STOP +/−) animals. Among the 16 litters of mice produced, none had a KO/KD pup that survived past two days postnatal. Of the eighty nine pups born in these litters 30 were wild-type, 34 were heterozygous KD, and 19 were heterozygous KO (χ2=25.72, p≦0.001). These results show that while the reduction of torsinA in homozygous KD mice is tolerable and can support perinatal development, an elimination of one of the KD alleles left an insufficient amount of torsinA to maintain survival. These results suggested that a threshold quantity of torsinA transcript between 45% and half that amount is necessary for survival.

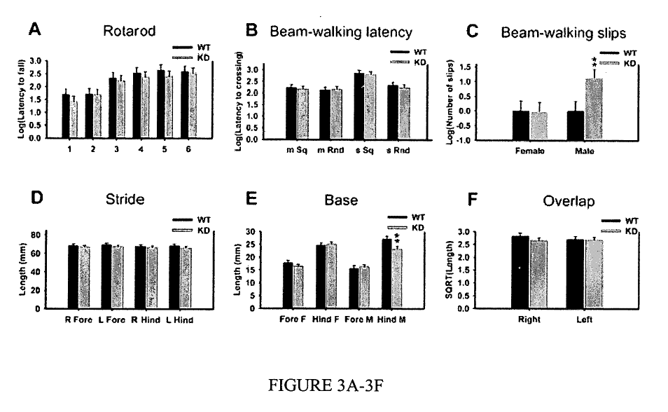

To determine if the reduction in torsinA interferes with motor development, the performance of Dyt1 KD mice were evaluated using a series of motor behavior tests. First, observations of body form were made and were followed by the rotarod, beam-walking, pawprint, and open-field tests. The first group was tested at three months old and the second at six months. For the initial test of hindlimb clasping, truncal posture and righting, the inventors used Fernagut and colleagues' assessment of body form in motor disorder. Each mouse was picked up by its tail and suspended for ten seconds to observe for hindlimb clasping. The mouse was then placed on the table for 1 min during which assessments of hindlimb extension and truncal arching were made. Finally, the mouse was tipped on its side by tail rotation and the ease of righting was noted. All mice demonstrated normal splaying of hindlimbs when suspended by their tails. Hindlimb extension and postural torsion were not detected. All mice displayed ease in righting when tipped onto their sides. For the rotarod test, an Economex accelerating rotarod (Columbus Instruments) was used. The apparatus started at an initial speed of 4 rpm and gradually accelerated at a rate of 0.2 rpm/sec. The latency to fall was measured with a cutoff time of 2 min. Both the WT and Dyt1 KD mice in the 6 months group were able to learn to walk on the rotarod and improved significantly over the course of six trials. There was no significant difference in latency to falling between WT and Dyt1 KD mice. Dyt1 KD mice at 6 months did not have a rotarod deficit (FIG. 3A).

Next, the mice were subjected to the beam-walking test. Mice were trained to traverse a square beam (14 mm wide). After training was completed, the experiment commenced with recordings of beam transversal time and number of hindpaw slips for each of the two trials per beam. On the first test day, animals were made to cross the medium square and round beam (17 mm diameter) and on the second day, a small round beam (10 mm diameter) and a small square beam (7 mm wide). As to the beam-walking tests for the six month group, the male Dyt1 KD mice displayed a significant increase of slips over their WT littermates (p=0.016, FIG. 3C). On average, male Dyt1 KD made nearly 218% more slips than WT mice (parameter estimate=e^{1.15±0.48}). Taken together, the Dyt1 KD mice displayed significant motor coordination and balance deficits in beam-walking test.

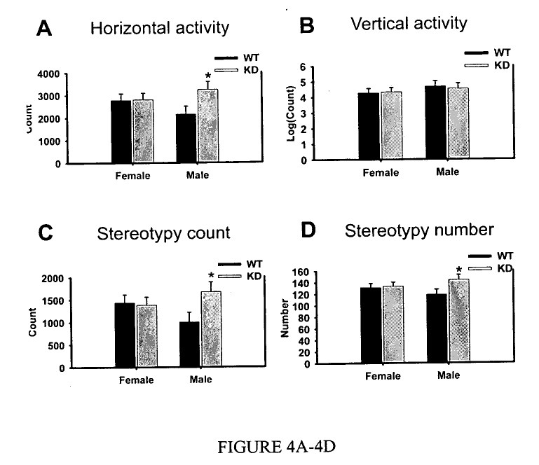

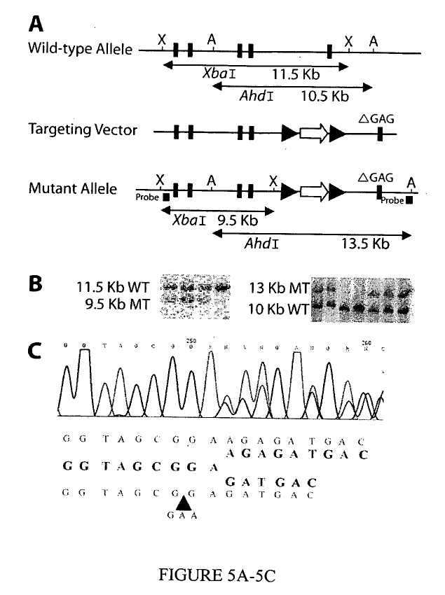

Finally, the mice were tested for their gait by pawprint analysis. For the six month group, there was no significant difference between WT and Dyt1 KD mice in stride length, forelimb base between Dyt1 KD and WT mice and in the hindlimb base between the female WT and Dyt1 KD mice. Male Dyt1 KD, on the other hand, showed a significantly smaller hindlimb base than WT mice hindlimb base (p=0.008; FIG. 4E). Taken together, male Dyt1 KD mice displayed an abnormal gait.

The six-month group was then analyzed for activity level in an open-field apparatus when they reached around 8 to 9 months of age. The statistical analysis of horizontal activity showed a significant difference between Dyt1 KD and WT male mice [p=0.037, FIG. 4A], with Dyt1 KD male mice more hyperactive than WT mice. Vertical activity between Dyt1 KD and control mice did not differ. Dyt1 KD male mice accumulated a significantly higher stererotypy count (p=0.038, FIG. 4C). This same difference is noticed in measurement of stereotypy number where the pairwise comparison between WT and Dyt1 KD male mice was significant (p=0.05). Taken together, the open-field data suggests that Dyt1 KD mice displayed hyperactivity with increased stereotypic activity, but had no difference in rearing behavior.

### Example 3

### Dyt1 ΔGAA (AE302) Knockin Mice

The Dyt1 ΔGAA mouse was generated using the same construct that was used for the knockdown (FIG. 2A and B). A re-screening of all the isolated ES cell colonies identified a total of three clones that contained ΔGAG mutation. These clones were used to generate a knockin ΔGAG line. Germline transmission was obtained from a few chimeric animals.

The heterozygotes named Dyt1 STOP ΔGAA (ΔE302) +/− that carried the ΔGAG mutation were interbred with CMV-cre mice [57]. In the progeny, the stop cassette was removed by cre-mediated recombination and mice that were heterozygous for Dyt1 ΔGAA (ΔE302) mutation (Dyt166 GAA +/− mice) were obtained. This genotype is the same as the patient affected by DYTI dystonia. The production of Dyt1ΔGAA mRNA was checked by sequencing the PCR amplification products that were reverse transcribed from mRNA isolated from Dyt1ΔGAA +/− mouse brains (FIG. 5C). As predicted, both wild-type and ΔGAG mRNAs were present.

Dyt1ΔGAA +/− mice were then bred to produce Dyt1ΔGAA −/− mice. Among the 153 pups analyzed, only 76 wild-type and 67 Dyt1ΔGAA +/− were present. The Dyt1ΔGAA −/− mice is therefore lethal (χ²=14.48, p≦0.001), similar to Dyt1 KO mice. The ΔGAG mutation is likely a haplo-insufficiency mutation.

The performance of Dyt1ΔGAA +/− mice was therefore evaluated using a series of motor behavior tests as described above for Dyt1 KD mice. One group of Dyt1ΔGAA mice was tested sequentially at 3 months and 6 months of age on a battery of motor behavioral tests described above.

At 3 months Dyt1ΔGAA mice did not show deficits in motor performance on any tests in comparison to WT mice. At six months, both WT and Dyt1ΔGAA had no observable hindlimb extension or truncal arching. When suspended from the tail, mice all have normal splaying of hindlimbs. Mice all exhibited strong righting reflexes when tipped on their side. Dyt1ΔGAA mice were able to complete the rotarod test successfully.

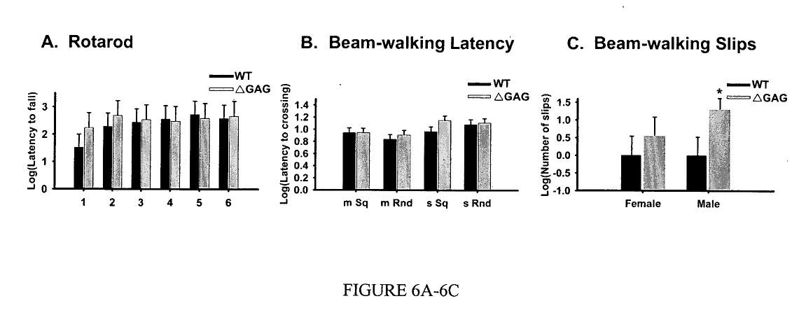

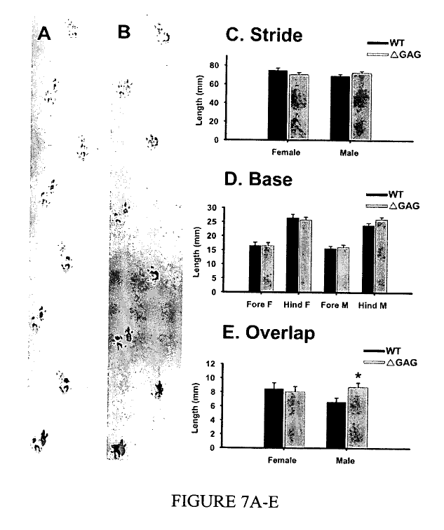

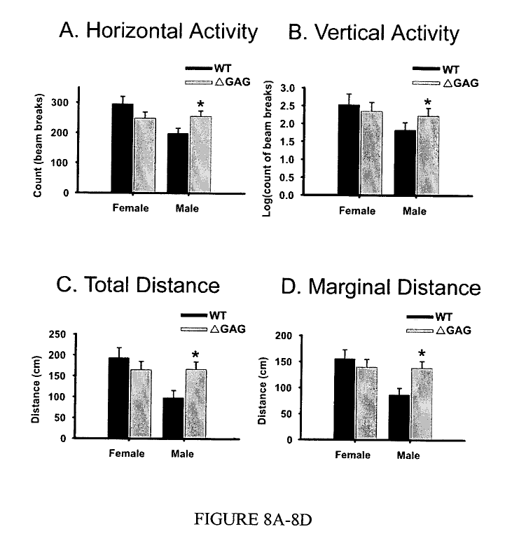

For the beam-walking test, the male Dyt1ΔGAA mice displayed a significant increase of slips over their WT littermates (p=0.013, FIG. 6C). On average, male Dyt1ΔGAA made nearly 215% more slips than WT mice (e^{1.15±0.48}). Two male Dyt1ΔGAA slipped, fell off at least one of the small beams, and were unable to complete the traversal. Taken together, the six-month-old male Dyt1ΔGAA mice displayed significant motor coordination and balance deficits in beam-walking test. In the gait analysis, mice did not exhibit obvious debilitating gait abnormalities (FIGS. 7A and B). Measurements of the stride lengths of fore -and hindpaws and the limb base analysis showed no significant differences between Dyt1ΔGAA and WT mice. The overlap measurement, however, did show a difference between Dyt1ΔGAA and WT, but only between the males (p=0.021). Male mice have a larger overlap in paw placements, thus showing an abnormality in gait. The motor activity test performed on the open-field apparatus revealed that Dyt1ΔGAA mice have hyperactivity which was mainly present in only male mutant mice (FIG. 8). Although not shown here, the inventors have also observed torsinA- and ubiquitin-positive aggregates in brainstem pontine nuclei in KI mice similar to DYT1 patients.

This Example demonstrates the generation of KI mice. These mice could serve as a mouse model of DYT1 dystonia useful for the development of therapeutic treatment. In this aspect of the invention. The KI mice may therefore be used in methods of screening for agents that alleviate the symptoms of dystonia by contact the KI mice with the test substance and determining characteristics such as motor coordination, balance e.g., using beam-walking tests as described above, or gait analysis, hindlimb extension or truncal arching characteristics, hyperactivity and other field activity characteristics as described in Example 2.

### Example 4

### Creation of RGS9L-cre Knock-in Mice and Efficient and Striatum-specific Gene Recombination

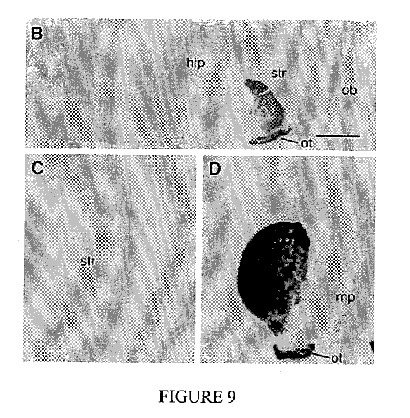

The striatum plays a major role in the circuits of neurological disorders. Until now, no striatal-specific cre mice have been available to target a transgene's expression specifically to this brain region. It was found that RGS9 gene, which codes for a regulator of G-protein signaling protein, has an alternatively sliced form (RGS9L or RGS9-2) that displays a highly restricted expression to the striatum [1,58,59] (FIG. 9). RGS9 gene turns on as early as embryonic day 16 in the striatum. RGS9 protein was initially characterized using directional tag PCR subtraction to isolate clones of rat striatum-specific mRNAs [60]. In situ hybridization, RNase protection assay, Western blot and immunohisto-chemistry have all indicated that expression of the RGS9L protein is highly enriched in striatum and is present in virtually all the medial spiny neurons in the striatum that carry the output of the striatum to GPi, GPe and substantia nigra. RGS9L protein is not present in globus pallidus. A knock-in approach similar to a previously published protocol [61], was performed to derive cre mice that would model the expression of RGS9L protein and mediate loxP-specific recombination restricted to the striatum.

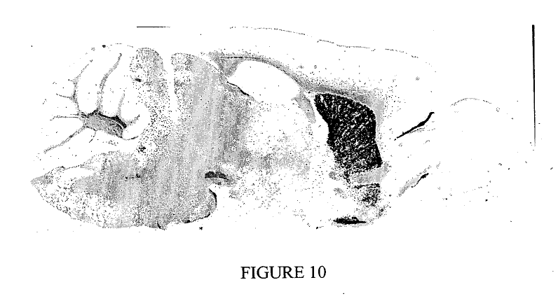

Mice positive both for RGS9L-cre and Rosa transgenes were identified using PCR. These mice (n=12; from P8 to P90) were perfused with fix solution and processed for β-galactosidase histochemistry as previous described [62]. Cre-mediated recombination was highly restricted to the striatum and nucleus accumbens. Recombination was also detected in the olfactory tubercle. A low level of recombination was also detected in the septal region, layers 5 and 6 of the cerebral cortex, the med preoptic nucleus, the periventricular hypothalamus, deep layers of the superior colliculus, the deep mesencephalic nucleus (FIG. 10). Overall, the recombination pattern is consistent with what has been published for RGS9L expression [1,58,59]. It should be noted that some of the scattered staining outside the striatum could be due to the axonal transport of non-nuclear localization of β-galactosidase. Thus, this example demonstrates the successful generation of a cre knock-in mouse that has RGS9L-specific expression of the cre gene. These results suggest that cre-mediated striatum- and nucleus accumbens-specific recombination is essentially complete by P8 and this strain can therefore be used to study the role of Dyt1 in striatal development and function.

### Example 5

### Generation and Preliminary Characterization of Tissue-specific Dyt1 Conditional Mutant Mice

As demonstrated above, the motor deficits and other phenotypes of KD and Dyt1ΔGAA mice were remarkably similar, suggesting loss- or reduction-of-function of torsinA is responsible for the pathogenesis in DYT1 patients. Therefore, these results support the use of conditional Dyt1 knockout mice to model DYT1 dystonia and study the contribution from different brain regions.

Striatum-specific Dyt1 KO (sKO) mice were produced from the crossing of Dyt1 loxP with RGS9L-cre mice. Cortex-specific Dyt1 KO (cKO) mice were generated by breeding the Dyt1 loxP with an Emx1-cre knock-in mouse that has been previously made in our lab [62]. In these mice, Cre expression is driven by an Emx1 endogenous promoter that has an expression pattern restricted to the cortex and hippocampus. Barrels, which are cortical representation of whiskers, in these mice were indistinguishable from control mice (n=5 each). Purkinje cell-specific Dyt1 KO (pKO) mice were derived from the crossing of Dyt1 loxP mice with Pcp2-cre mice [63]. All sKO, cKO, and pKO mice had both copies of Dyt1 gene inactivated in their tissue-specific, cre-expressing brain regions and were in mixed genetic background. They were born in a Mendalian fashion and survived to adulthood. Since both KD and Dyt1ΔGAA mice showed behavioral deficits characterized as increased slips in beamwalking tests and hyperactivity in open-field tests, our preliminary analysis of conditional knockout mice was limited to these two tests.

A total of 60 mice were tested for cKO batch, with 25 Dyt1 loxP heterozygous or homozygous mice (L mice), 18 Dyt1 loxP/deleted mice (LD mice) and 17 cKO mice. LD mice had one copy of Dy1 gene already recombined and only had one functional copy of the Dyt1 gene. LD mice were likely express only 50% of Dyt1 mRNA, close to the KD mice that expressed 45% of Dyt1 mRNA. These mice were 107 to 210 days postnatal at the beginning of the behavioral experiments.

A total of 34 mice were used for sKO batch with 13 sKO mice, 13 Dyt1 loxP heterozygous or homozygous mice (L mice) that served as alternatives to wild type mice, and 8 mice (DHET) that were heterozygous both for Dyt1 loxP and RGS9L-cre transgenes. DHET mice therefore had one copy of Dyt1 gene inactivated in the striatum. These mice were 141 to 184 days postnatal at the onset of the behavioral testing.

A total of 66 mice were tested for pKO batch, with 32 Dyt1 loxP heterozygous or homozygous mice (L mice), 17 Dyt1 loxP/deleted mice (LD mice) and 17 cKO mice. LD mice had one copy of Dy1 gene already recombined and only had one functional copy of the Dyt1 gene. LD mice were likely express only 50% of Dyt1 mRNA, close to the KD mice that expressed 45% of Dyt1 mRNA. These mice were 107 to 233 days postnatal at the beginning of the behavioral experiments.

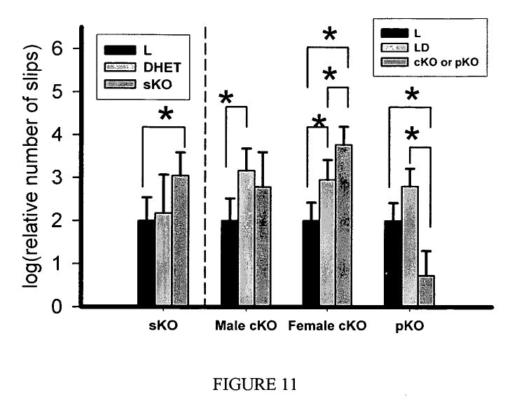

Beamwalking test: For sKO batch, although there was no statistically significant difference between the DHET and L mice, regardless of the gender, the sKO mice showed 186% more slips than L mice in beam-walking tests (p=0.05; parameter estimate=e^{1.15±0.48} FIG. 11).

For cKO batch, there was a significant interaction among genotype, sex, and trial (p=0.03). Detailed analysis revealed that the female cKO mice had 130% more slips than the control female LD mice (e^{0.81±0.42}, p=0.04; FIG. 11), while the difference between male cKO and LD or L mice was not significant (p=0.8). Interestingly, as predicted from the studies conducted on KD mice, regardless of sex, LD mice showed about 220% more slips than L mice (p=0.024). In conclusion, female cKO mice showed a beam-walking deficit. It may be that the onset of a deficit could be delayed in male cKO mice this can be tested upon breeding with the C57BL6 background.

For pKO batch, surprisingly, the pKO mice showed significantly much less slips than the L mice (p=0.012, e^{-1.27±0.42}=28%), i.e., L mice on average made 260% more slips than pKO mice. As demonstrated in cKO batch, the LD mice showed about 123% more slips than L mice (p=0.05).

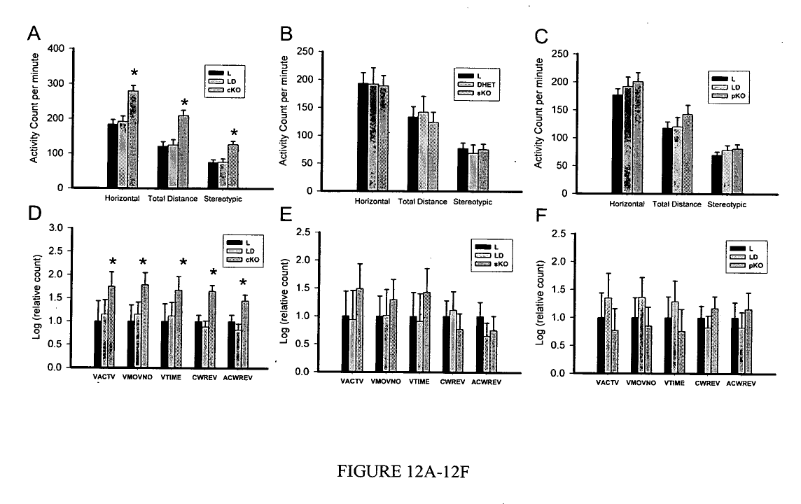

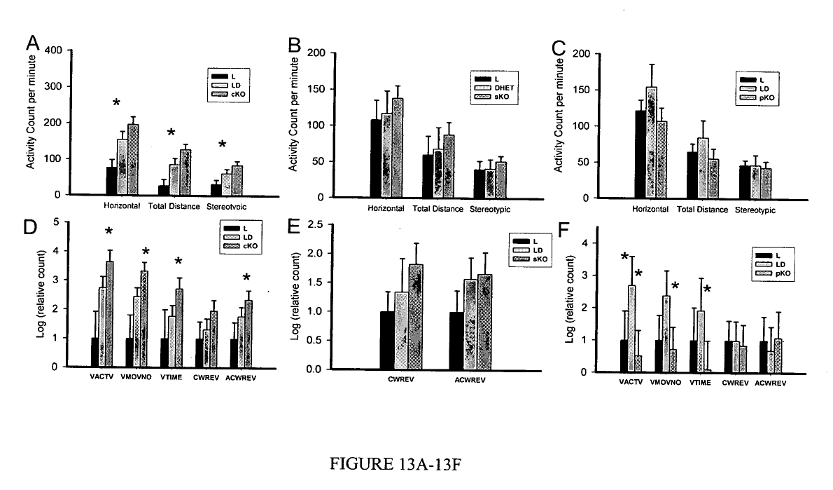

Open-field test: For sKO batch, there were no statistically significant differences between sKO and their control littermates (DHET and L mice; FIGS. 12B, 12E). Tests of the same batch of the mice 3 months later still failed to detect any hyperactivity in sKO mice (FIGS. 13B, 13E).

For the cKO mice, regardless of the gender, the cKO mice were hyperactive as indicated by significantly increased horizontal, vertical (VACTV) and stereotypic activities. The cKO mice had significantly larger value of total distance traveled and increased circling activities (CWREV and ACWREV; FIGS. 12A, 12D). Furthermore, the cKO mice also had significantly more vertical movement number (VMOVNO), more circling, and spent more time on vertical movements (VTIME). The same mice tested 3 months later showed similar results, except this time, LD mice showed more hyperactivity as predicted from the results obtained from Dyt1 KD and Dyt1ΔGAA mice (FIGS. 13A, 13D).

For pKO mice, there is no significant difference between conditional knockout mice and their control L mice. Repeated tests at 3 months later again failed to detect any hyperactivity (FIGS. 12C, 12F, 13C, and 13F).

Taken together, conditional inactivation of Dyt1 gene either in cerebral cortex/hippocampus or striatum led to significantly more slips in beam-walking tests. While Purkinje cell-specific inactivation of Dyt1 gene led to the significant improvement of the performance on the beams, suggesting the effect of Dyt1 gene inactivation on beam-walking slips is highly cell type-specific. Furthermore, it was shown that inactivation of Dyt1 gene in cerebral cortex and hippocampus, but not in striatum and Purkinje cells, could lead to hyperactivity in open-field tests.

### Example 6

### Identification of a Short Hairpin RNA That Can be Used for the Silencing of Both Human and Mouse torsinA Gene

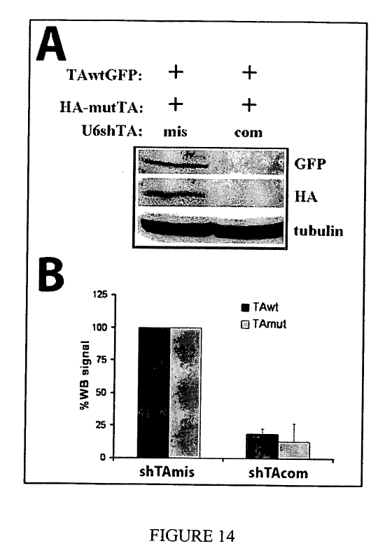

As shown above, improvement in motor coordination in pKO suggest a possible strategy of gene therapy for DYT1 dystonia through Purkinje cell-specific silencing of DYT1 gene. A shRNA (shTAcom) has been developed that was based on the common sequence between mouse and human torsinA gene (nucleotides 931-969 of SEQ ID NO: 1) and is set out as SEQ ID NO: 3 (CAGTGGCTTCTGGCACA GCAGC). It was effective in human cells (FIG. 14).

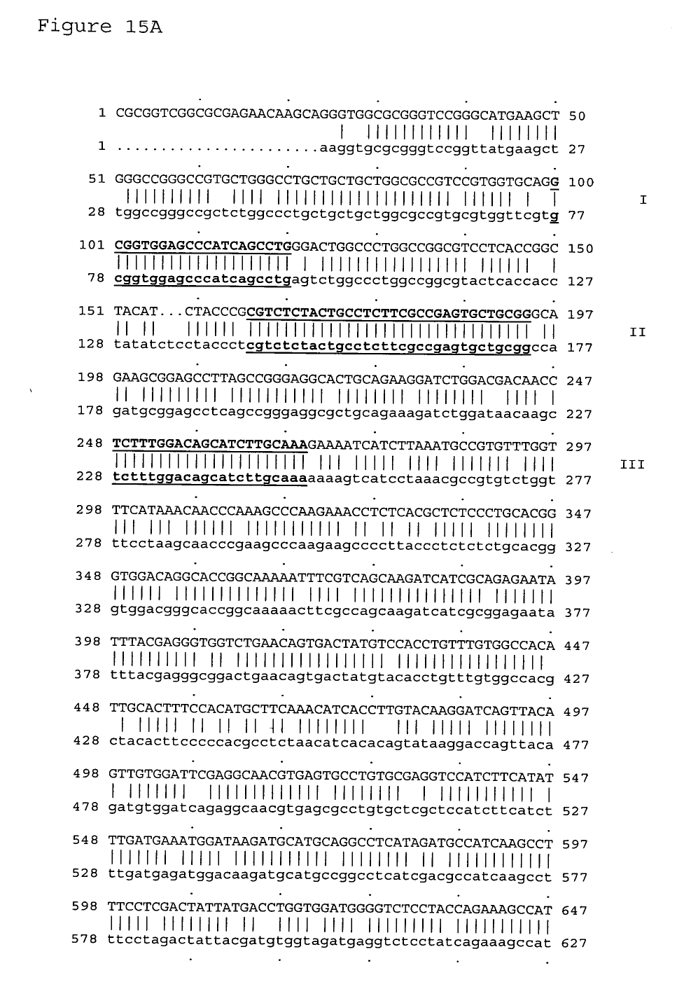

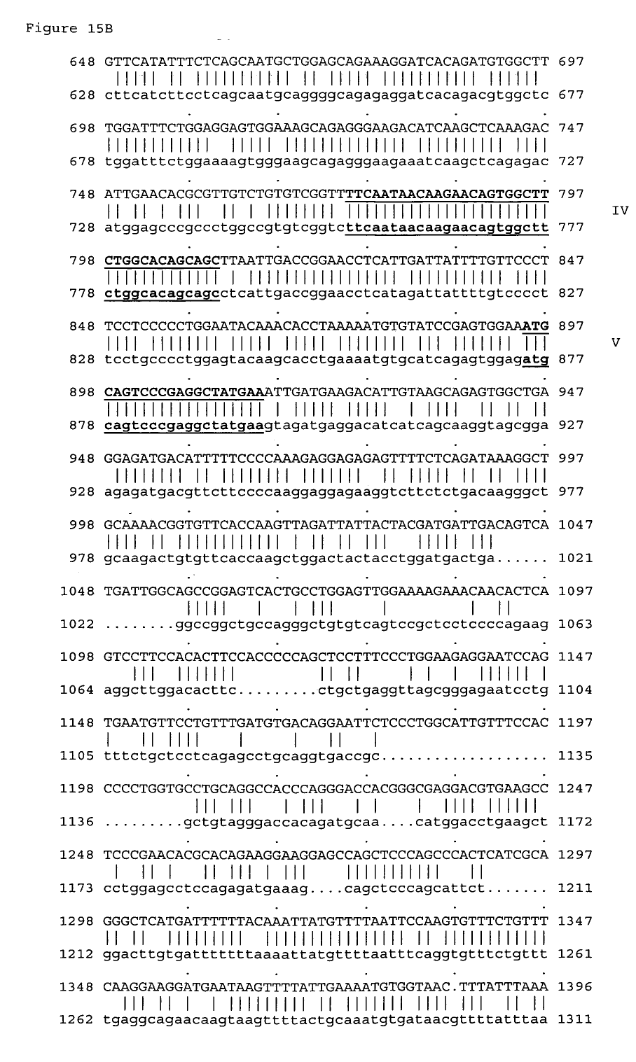

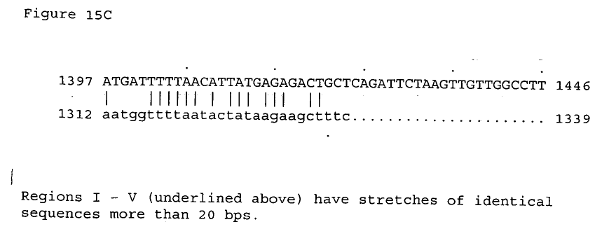

Additional shRNA sequences that are effective for silencing the DYTI gene in the methods of the invention may be identified using the common sequence between mouse and human torsinA gene. This common sequence was identified by aligning the mouse and human torsinA polynucleotide sequence as provided in FIG. 15. shRNA sequences were designed based on stretches of identical sequence of more than 20 nucleotides. Exemplary shRNA sequences identified by this technique are those set out as SEQ ID NO: 4-14. The sequences include:

5′ CAGGCUGAUGGGCUCCACCGC 3′
5′ ACUCGGCGAAGAGGCAGUAGAGACG 3′
5′ CCGCAGCACUCGGCGAAGAGGCAG 3′
5′ UUUGCAAGAUGCUGUCCAAAGA 3′
5′ AGAAGCCACUGUUCUUGUUAUUGAA 3′
5′ GCUGCUGUGCCAGAAGCCACUGUUC 3′
5′ UUCAUAGCCUCGGGACUGCAU 3′

### Example 7

### Analysis of Motor Coordination and Balance in Purkinje Cell-specific Dyt1 Knockout/Dyt1ΔGAG Knock-in Double Mutant Mice

The results presented in Examples 1 through 6 show that Purkinje cell-specific Dyt1 knockout mice have dramatically improved motor coordination and balance skills when performing the beam walking task. In the present Example, the aim is to explore whether the same mutation introduced to the Dyt1ΔGAG knock-in mice would show the same benefit and correct the motor coordination and balance deficits exhibited by Dyt1ΔGAG knock-in mice (FIG. 6C). In order to achieve this aim the pKO mice (genotype Pcp2-cre+/−Dyt1loxP−/−) will be bred with Dyt1ΔGAG knock-in mice (heterozygous; homozygous are lethal). Four genotypes will be produced in equal ratio: Set A) Pcp2-cre+/−Dyt1loxP/ΔGAG, Set B) Pcp2-cre+/−Dyt1loxP+/−, Set C) Dyt1loxP/ΔGAG, and Set D) Dyt1loxP+/−. In Set A mice, pKO mutation has been introduced to Dyt1ΔGAG knock-in background and these mice therefore will be experimental animals. Set B mice will control the effect of Pcp2-cre transgene but will have one copy of Dyt1 inactivated in Purkinje cells. The data from Set B mice can be compared with the data from Set D to see whether this would have an effect. Set C mice are very similar to Dyt1ΔGAG knock-in heterozygous mice, since the inventors have not detected any undesirable effect of inserting two short, 50 bp loxP sequences in introns 2 and 4, respectively. Set D mice are equivalent to wild type mice and Set B and Set D mice will serve as controls. The motor development and behaviors of all 4 genotypes of mice will be compared. If Set A mice show significantly improved performance over Set C mice this would demonstrate that Purkinje cell-specific silencing of Dyt1 expression improves motor performance in Dyt1ΔGAG knock-in mice. The following is a more detailed description of how such studies will be performed.

Animals: 10 Male and 10 female mice will be used for each genotype unless noted in the specific procedure. Therefore, for each experiment, a total of 80 mice will be tested. 4 Different age groups will be tested at the following ages: P90, P180, P270, and P365 (P: postnatal day).

It is well established that mouse behavior is significantly influenced by the strain background of the mouse [64-66]. Behavioral analysis have shown variable performance levels among wild-type animals of different inbred strains [67,68]. This characteristic strain difference is especially relevant to movement disorder models. For example, C57/BL6 and CBA have been reported to outperform other strains tested on all motor behavioral tests, while strains such as 129/Sv perform least well on many of the tests [69]. Due to the uncontrollable consequence of making a genetically-altered mouse using gene targeted stem cells, our mice currently have genetic material from a mixture of backgrounds. The inventors will use mutant animals backcrossed more than 6 generations into C57BL6 background by the time of the review of this proposal. Strain contribution to behavioral tests is an important consideration.

Methods and Procedures: First the biochemical and immunohistochemical experiments will be performed to determine the specificity and efficiency of Purkinje cell-restricted Dyt1 inactivation. In situ hybridization will be used as one approach to confirm that Dyt1 mRNA has been deleted in the Purkinje cells. It will be done according to the published procedures [70,71] on frozen 12-μm coronal sections of mouse brains. Brains from 5 Set A mice and 5 Set D animals (aged from P28 to P60) will be dissected out and quickly frozen in isopentane. Coronal sections will be obtained from these brains using a cryostat. The sections will be fixed in 4% formaldehyde/saline for 10 min. Digoxigenin-labeled probes will be generated in both transcription directions by using a subclone in the Bluescript vector (Stratagene) containing cDNA corresponding to exons 3 to 5 of the Sgce gene. Sections will be hybridized at 53° C. for 24 hrs in a solution containing 50% formamide and a digoxigenin-labeled RNA probe in either sense or antisense (a sequence complementary to mRNA) direction. Unbound probes will be removed by a serial of washings (final washing with 0.1×SSPE/1 mMDTT at 65° C.). The section will be further treated by alkaline phosphatase-conjugated antibodies to digoxigenin overnight with gentle shaking at room temperature. The bound probes will be finally visualized by incubating in NBT/BCIP substrate working solution overnight. The sections will then be washed in 1×SSPE several times, dried and coverslipped.

Immunohistochemical localization of torsinA proteins will be used to confirm the deletion of Dyt1 gene in Purkinje cells. This will be carried out according to the published protocols [71]. Briefly, 50 μm coronal forebrain sections from 5 Set A and 5 Set D animals (about 4 to 8 weeks old) will be cut with a freezing microtome, collected in PBS, and preincubated in TBS (Tris-buffered saline) +5% normal animal serum (NAS) for 30 minutes at 4° C. with shaking. The sections will then be incubated overnight at 4° C. with shaking with primary antibodies in TBS+0.1% NaN3+5% NAS. The sections will be rinsed three times with TBS and incubated in biotinylated secondary antibody for 2 hours at 4° C. with shaking. Finally, the sections will be treated using the Vectastain ABC kit. Antibodies to torsinA proteins are commercially available from Santa Cruz Biotechnology, Inc.

The number of Purkinje neurons positive either for Dyt1 mRNA or torsinA protein will be quantified using Stereo Investigator software and a stereology workstation in the Beckman Visualization facility (http:\\itg.beckman.uiuc.edu).

Behavioral characterization and statistical analysis will be performed using the motor test battery as outlined above.

Anticipated results from the above studies: It is expected that the Set A mice will have Dyt1 gene inactivated in over 90% of the Purkinje cells. As to the behavioral tests, it is predicted that the Set A mice will show motor improvement over Set C mice. The improvement will likely be in the form of: 1) significant less slips during beam-walking tests, 2) normal gait as determined by pawprint test. There may also optionally be an improvement in hyperactivity in the open field.

To determine whether Purkinje cell-specific knockout of Dyt1 gene had any harmful effect, the performance of the pKO mice in rotarod and pawprint tests was measured. The same batch of mice was used for these two tests. At the time of rotarod testing, the mice were about 252 days old. The test was done over two days with 3 trials each day as described (Dang et al., 2005, Exp Neurol 196, 452-463). There was no genotype and trial interaction [F(5,155)=0.89, p=0.4861]; therefore, the latency was estimated with 6 trails combined. There was no significant difference between pKO and L mice [F(5,155)=0.89, p=0.4861] suggesting pKO mice showed no rotarod deficits. The latency data were also estimated by each trial.

The pKO mice were then analyzed for their gait using pawprint analysis. At the time of testing, the mice were about 227 days old. There was no significant two- or three-way interaction detected. As listed in the following table, no gait abnormalities were detected in pKO mice.

| Genotype | Stride (mm) | Overlap (mm) | Base (mm) |
|---|---|---|---|
| pKO mice | 75.1557 ± 1.4742 | 9.2359 ± 0.6857 | 20.3736 ± 0.6068 |
| L mice | 74.4744 ± 1.1181 | 9.2618 ± 0.5208 | 20.4990 ± 0.4602 |
| P value | 0.2881 | 0.8163 | 0.2105 |

The morphology of the Purkinje cells and other associated cerebellar structures of pKO mice with electron microscope also was examined. The pKO mice showed normal Purkinje cells with intact nuclear membranes. The cytoplasmic structures and contents were similar and showed no abnormalities. Underneath the Purkinje cell layers, the granule cells were seen tightly packed and showed normal nuclear membrane structures. On the other side of the Purkinje cell layers are molecular layer. At the low magnification of about 2,000X, both axons and dendrites were seen with normal diameters and packing densities. At higher magnification (50,000X), boutons were clearly visible as well as postsynaptic density. Thus synapse formation in molecular layers appeared to be normal in both control and knockout mice.

Taken together, the analysis using rotarod, pawprints analysis, and ultrastructural examination did not detect any harmful effects resulted from Purkinje cell-specific Dyt1 knockout. These results support the use of the Purkinje cell-specific knockout of Dyt1 to cure the motor deficits in Dyt1ΔGAG knock-in mice.

The results presented above demonstrated that Purkinje cell-specific Dyt1 knockout mice showed dramatically improved motor coordination and balance skills when performing the beam walking task. The following data explored whether the same mutation introduced to the Dyt1ΔGAG knock-in mice would show the same benefit and correct the motor coordination and balance deficits exhibited by Dyt1ΔGAG knock-in mice. pKO mice (genotype Pcp2-cre+/−Dyt1 loxP−/−) were bred with Dyt1ΔGAG knock-in mice (heterozygous; homozygous are lethal). Four genotypes were produced in equal ratio: A) Pcp2-cre+/− Dyt1loxP/ΔGAG, B) Pcp2-cre+/−Dyt1loxP+/−, C) Dyt1loxP/ΔGAG, and D) Dyt1loxP+/−.

In A type mice, pKO mutation had been introduced to Dyt1ΔGAG knock-in background and therefore was experimental animals. B mice should control the effect of Pcp2-cre transgene but had one copy of Dyt1 inactivated in Purkinje cells. This should not be a problem since it should possible to compare the data from B with D to see whether this would have an effect. C mice are very similar to Dyt1ΔGAG knock-in heterozygous mice, since there was no detection of any undesirable effect inserting two short, 50 bp loxP sequences in introns 2 and 4, respectively. D mice are equivalent to wild type mice and B and D mice served as controls. The first batch of mice used had the sex and genotype distributions shown in the following table.

| Genotype | Male | Female | Total |
|---|---|---|---|
| A mice: Pcp2cre+/− Dyt1 ΔGAG/loxP | 8 | 3 | 11 |
| B mice: Pcp2-cre+/−Dyt1 loxP | 5 | 5 | 10 |
| C mice: Dyt1 GAG/loxP | 8 | 7 | 15 |
| D mice: Dyt1 loxP+/− | 6 | 1 | 7 |
| TOTAL | 27 | 16 | 43 |

At the onset of the behavioral testing, the mice were 157 days to 171 days old, with an average of P166. There were no significant age and bodyweight differences among the four genotypes. The experimenters were blind to the genotypes. Littermates from 10 litters were genotyped and pooled together to form this batch.

Motor coordination and balance was analyzed using beam-walking test. Mice were trained for two days to traverse a square beam (14 mm wide). After training was completed, the experiment started with recording of numbers of hindpaw slips for each of the two trials per beam. On the first test day, animals were made to cross the medium square (MS) and round beam (MR) (17 mm diameter) and on the second day, a small round beam (SR; 10 mm diameter) and a small square beam (SS; 7 mm wide). First, 3 male and 1 female C mice (similar to Dyt1ΔGAA mice) fell off the SR or SS beam during their tests on small beams. These mice were assigned maximum number of slips (slip=16) for the trials that they fell. None of the A mice fell off the beams. The rescue effect of Purkinje cell-specific Dyt1 inaction on Dyt1ΔGAG knock-in mutation approached significance (p=0.11, Fisher's exact test, two tails).

Next, analyses were carried out to determine whether there is any difference in the numbers of slips between B and D mice. The data from 10 B type and 7 D type mice were pooled and analyzed by logistic regression (GENMOD). No significant difference was seen between the B and D mice (p=0.3146). One copy of the Dyt1 gene is inactivated in the Purkinje cells of B mice. The B mice are not ideal control mice for the C and D mice; therefore, the following analysis was performed without B mice.

First, the accumulated distribution of the numbers of slips of the A, C, and D mice were plotted. C mice showed much more slips than the D mice. It was significant that that as predicted above, when Dyt1 gene was inactivated in Purkinje cells, the distribution of the A mice shifted significantly toward the D mice. In fact, the A mice performed as well as the D mice (see statistics below).

Detailed analysis using logistic regression (SAS software version 9.1, GENMOD with GEE model) indicated that although there was a trend of genotype and beam interaction (DF=6, χ2=12.25, p=0.0567), the interaction did not reach the significance. The slip data from the 4 beams were combined and analyzed together. As expected from the predictions presented above, and regardless of genotype, there was a statistical significance of beam types (DF=6, χ2=12.69, p=0.0054). The SS beam showed the most slips while MS beam showed the least slips since it was used as a training beam for the first two days. The effect of genotype also reached significance. Similar to what was reported previously (Dang et al., 2005), the C mice showed 202% (el.1042-1; p=0.0295) more slips than the D mice, suggesting the beam walking deficit of Dyt1 knockin mice is very robust and highly reproducible. The C mice also showed 121% (e0.7943-1; p=0.0421) more slips than the A mice. Both of the increases reached significance. There was no difference between the A and D mice in this test (p=0.5295).

Taken together, these data show that Purkinje-cell specific Dyt1 knockout rescued motor coordination and balance deficits normally associated with ΔGAG in the Dyt1 gene.

In additional studies, in situ hybridization was used to demonstrate Purkinje cell-specific Dyt1 deletion. A 351 bp fragment was cloned from 3′-untranslated region of the torsinA mRNA into pGEN(+) plasmid (Promega) and DIG-labeled complementary RNA probes were prepared using SP6 RNA polymerase using the labeling kit from Roche. The probes were hybridized overnight at 55° C. to sagittal sections (30 μm) from both the Dyt1 loxP−/− and pKO mice (n=1) each. The hybridization solutions contained 50% formamide, 4×SSC, 1×Denhardt, salmon sperm ssDNA, and yeast tRNA. The unhybridized cRNA probes were then removed by RNaseA digestion and washed with 1 time each with 5×SSC, 1×SSC, or 0.1×SSC at 60° C. followed by 50% formamide, 2×SSC at 50° C.

The bound probes were then reacted with alkaline phosphatase-labeled antibody (Roche Neucleic Acid detection kit). Similar to what has been published (Xiao et al., 2004 Brain Res Dev Brain Res 152, 47-60), high levels of mRNA were detected in the Purkinje cell (PC) layer of the Dyt1 loxP−/− mouse but not in pKO mice. Taken together, these studies show that the inventors have successfully demonstrated the specificity of Pcp2-cre mediated deletion. Similar specificity has been demonstrated with FMR1 (NEURON 47: 339-352, 2005), cGMP-dependent protein kinase I (J. CELL BIOLOGY 163: 295-302,2003), calbindin (J. OF NEUROSCIENCE 23: 3469-3477, 2003), Calb1 (GENESIS 32 (2): 165-168, 2002) genes. Pcp2 promoter qualifies to direct Purkinje cell-specific recombination.

### Example 8

### Construction of AAV Vector and Analysis of Motor Coordination and Balance in Dyt1ΔGAG Knock-in Mice After Intracerebellar Delivery

AAV is safe to use and has been the choice for a few current clinical trials and animal models of neurological disorders [54]. Recently, AAV-based shRNA has been used to suppress the polyglutamine-induced neurodegeneration in a mouse model of spinocerebellar ataxia type 1 (SCA1) [55]. SCA1 mice contain a transgenic human disease allele (ataxin-1-Q82) controlled by Purkinje cell-specific PCP-2 promoter. The shRNA against human SCA1 sequence was used to successfully down-regulate the expression of transgene without any effect on endogenous mouse Sca1 gene. Upon intracerebellar injection of the AAV virus that expresses the shRNA against SCA1 sequence, improvement in motor coordination, restored cerebellar morphology, and resolved characteristic ataxin-1 inclusions were achieved. The same group also succeeded in achieving improvement in motor performance and neuropathological phenotypes in a Huntington's disease mouse model using similar strategies [56].

In the present Example, studies are designed to use an AAV backbone plasmid to construct the following AAV vector:

ITR—mouseU6 promoter—TATA-lox—TTTTTT-Pcp2 promoter—cre—TATA-lox—shTAcom—TTTTTT—CMV promoter—AcGFP1—SV40polyA—ITR.

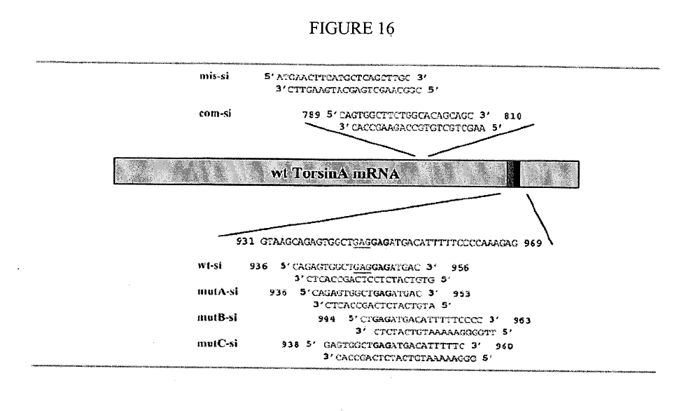

The development of the shTA is described by Gonzalez-Alegre et al., (Ann Neurol 2003; 53:781-787). The exemplary sequence from that reference are shown in FIG. 16, which is an excerpt of FIG. 2 from the above reference.

Mouse U6 promoter, shTAcom, and shTAmis will be provided by collaborator Dr. Pedro Gonzalez-Alegre of University of Iowa (see FIG. 14 and page 41). TTTTTT strings serve to terminate transcription initiated by U6 promoter. TATA-lox is a modified loxP site developed by Ventura and colleagues [52] for cre-loxP-regulated RNA interference. A TATA box has been constructed in this loxP sequence. Pcp2 promoter (1 kb) will be cloned from mouse genomic DNA directly using high fidelity PCR and verified by sequencing [72]. A modified cre [62] constructed in my lab that has nuclear localization signal will be fused in frame to the start codon of the Pcp2 protein. CMV promoter-AcGFP1-SV40polyA cassette (1.6 kb) will be excised from pAcGFP1-C1 commercially available from Clontech (BD Bioscience). The total length of the construct will be about 4.6 to 4.7 kb. Before cre-mediated recombination and in infected cells, a short RNA will be produced from U6 promoter containing the left TATA-lox sequence and terminating by the first 6 Ts.

Upon packaging and intracerebellar injection, only infected Purkinje cells will express Cre protein and will recombine 2 TATA-lox into one TATA-lox and delete the TTTTTT-Pcp2-cre sequence. The vector is then reduced to: ITR-mouseU6 promoter—TATA-lox—shTAcom—TTTTTT—CMV promoter—AcGFP1—SV40polyA—ITR

This would enable the U6 mediated transcription to produce shTAcom RNA to silence the expression of Dyt1 gene on Purkinje cells. AcGFP has been optimized to the mammalian codon and should serve as controls to determine how effective the infection could be after intracerebellar injection.

Another control AAV vector in which the shTAmis replaces shTAcom in the above construct (FIG. 14) will be prepared to serve as a control for vector infection, cre expression, and other factors.

Methods and Procedures: DNA cloning, PCR, and vector construction are well established and will be performed according to standard protocols. The vector will then be sent to Dr. Miguel Sena-Esteves of MGH/Harvard Medical School, who has been Director of the MGH Neuroscience Center Vector Design and Development Core since 2003 in order to prepare the AAV virus particles at the direction of the present inventors. Either serotype 1 or 5 will be used. Both of these serotypes have shown tropism for Purkinje cells [55, 73]. In fact the published experiments of SCA1 mice were performed using AAV1 serotype.

Intracerebellar injection of AAV virus particles and their validation: Eight-week old male Dyt1ΔGAG mice and wild type male littermates will be anesthetized with avertin. Male mice are preferable to female mice because of their earlier expression of motor deficits (FIG. 6). A burr hole will be drilled at the midline posterior occipital bone overlying the cerebellar anterior lobe. Pressure injections (2 μl total) will be made into a single cerebellar lobule using a Hamilton syringe connected to a disposable glass micropipette tip. A total of 20 mice (10 mice of each genotype) will be injected with AAV virus particles for each experiment. Animals will be sacrificed at 3-6 weeks after gene transfer and cerebella will be removed. Tissues will be fixed in 4% paraformaldehyde overnight at 4° C., cryoprotected for 1 day in 30% sucrose in phosphate buffered saline at 4° C. and then sectioned sagitally at 50 μm. GFP florescence will indicate the area infected by rAAV virus. Immunohistochemistry and in situ hybridization protocols described in Aim 1 will also be used to assess the success of the Purkinje cell-specific silencing.

Use of Rosa indicator mice to determine specificity of gene transfer: As an alternative to assess the efficiency of Pcp2-cre mediated recombination, the AAV virus particles will also be injected as described above to Rosa mice [74], a line of loxP indicator mice that have been used previously [62] (see FIG. 10). If Pcp2-cre mediated recombination is successful, Purkinje cells will be stained blue when x-gal substrate is applied to the brain sections. The use of the Rosa mice will also help to assess how specific the cre will be expressed under the control of the Pcp2 promoter. Ectopic expression of cre outside of Purkinje cells will be detected by staining for β-galactosidase. Staining and processing of brain sections will be done according to the published paper from PI's lab [62].

Large scale injection of AAV virus and testing of motor behaviors: After the intracerebellar injection procedure is established, a total of 5 batches (20 mice each mutant and wild type mice per batch) of Dyt1ΔGAG and wild type male mice in C57BL6 background will be prepared. Only one AAV injection session will be done for each batch at the age of P35, P60, P120 or P150. Half of the mice will be injected with virus expressing shTAcom and the rest will be infected with virus that will express shTAmis. The mice will be allowed to survive and the motor behavior including motor coordination and balance will be tested starting at P180 (6 months old).

Behavioral characterization and statistical analysis will be performed-using the motor test battery as described above.

Neuropathology examination: The torsinA- and ubiquitin-positive aggregates in the pontine nuclei of the brainstem will also be determined. The inventors have demonstrated only Dyt1ΔGAG mice showed such aggregates. The aggregates will be stained according to techniques known in the art.

Anticipated results: the present Example is expected to demonstrate that there is an improvement of motor performance in beam walking test. The Dyt1ΔGAG mice treated with AAVshTAcom will show significantly less slips than the Dyt1ΔGAG mice without treatment or treated with AAVshTAmis. AAVshTAcom-treated Dyt1ΔGAG mice will likely also show an improvement in gait. While an improvement of hyperactivity in the open field test and reduction of torsinA- and ubiquitin-positive aggregates is not necessarily expected, it may be observed; however, the inventors these deficits are of cortical, striatal, or both origins. It should be noted that slip deficits in beam walking is the most prominent and consistent motor deficits in the dystonia mice the inventors have analyzed as well as to the other mouse model of movement disorders such as Parkinson disease [75]. The improvement of slip deficits should amount to a major advance in the field of development of RNAi therapy using animal models of movement disorders. While the initial therapeutic protocols will employ AAV, the inventors also will switch to lentivirus or other vectors that allow for much larger insert that will improve the fidelity of Pcp2 promoter.

### Example 9

### Further Characterization of Dyt1ΔGAG Knock-in Mouse as a Model for Early-onset Dystonia

The examples presented herein above show the generation and characterization of a gene-targeted mouse model of Dyt1ΔGAG to mimic the mutation found in DYT1 dystonic patients. The mutated heterozygous mice had deficient performance on the beam-walking test, a measure of fine motor coordination and balance. In addition, they exhibited hyperactivity in the open-field test. Mutant mice also showed a gait abnormality of increased overlap. Mice at 3 months of age did not display deficits in beam-walking and gait, while 6-month mutant mice did, indicating an age factor in phenotypic expression as well. While striatal dopamine and 4-dihydroxyphenylacetic acid (DOPAC) levels in Dyt1 DGAG mice were similar to that of wild-type mice, a 27% decrease in 4-hydroxy, 3-methoxyphenacetic acid (homovanillic acid) was detected in mutant mice. Dyt1 DGAG tissues also have ubiquitin- and torsinA containing aggregates in neurons of the pontine nuclei. A sex difference was noticed in the mutant mice with female mutant mice exhibiting fewer alterations in behavioral, neurochemical, and cellular changes. Further data to support the above conclusions are presented in the instant example. The results of these studies show that knocking in a Dyt1 DGAG allele in mouse alters their motor behavior and recapitulates the production of protein aggregates that are seen in dystonic patients. In addition, the data further support alterations in the dopaminergic system as a part of dystonia's neuropathology.

Motor behavioral tests were performed as described above and included The test battery consisted of body form assessment, rotarod, beam-walking, and pawprint tests and was performed in four consecutive weeks with one test performed each week in the above order. One month after completion of these tests, the 6-month group was tested in the open-field analysis.

In addition, HPLC dopamine and metabolite measurements were taken: The protocol devised to measure dopamine (DA) and its metabolites, 4-dihydroxyphenylacetic acid (DOPAC) and 4-hydroxy-3-methoxyphenylacetic acid [homovanillic acid (HVA)], was based on several sources [76; 77; 78]. For striatal tissue dopamine/metabolite measurements, striata were dissected from Dyt1 ΔGAG and WT male and female littermates of around 11 months old (n=14 Dyt1 ΔGAG, n=15 WT). Striata were homogenized in ice-cold 0.2 N perchloric acid (5 μl/mg tissue) and centrifuged for 15 min at 15,000×g at 4° C. to remove debris. Twenty microliters of the supernatant representing 2 mg of tissue, was then applied to a C18 reverse phase HPLC column (Varian) connected to an ESA model 5200A electrochemical detector. The running buffer used was 50 mM potassium phosphate buffer with 0.5 mM octyl sulfate and 5% acetonitrile. One-way ANOVA was used to analyze the quantities and ratios detected in Dyt1 ΔGAG and WT groups. Means and standard errors were obtained using the Tukey's HSD method.

Brain Histology: Mice were heavily anesthetized with pentobarbital and perfused with phosphate buffer followed by 4% paraformaldehyde. Brains were dissected and soaked in 4% paraformaldehyde overnight and then in 30% sucrose in phosphate buffer foran additional night. Brains were sectioned in the coronal plane at 50 Am thickness using a freezing sliding microtome and processed with thionin-based Niss1 stain as described previously [79]. The sections were mounted, stained, and coverslipped with DPX. Dried slides were scanned using a Nikon medical slide scanner linked to a computer. Highresolution images were captured using a video camera controlled by Stereo Investigator (MicroBrightfield, Inc.) software.

Immunohistochemistry: The protocol was described elsewhere [80]. Briefly, mice 6 months old (n=6 Dyt1 ΔGAG, n=5 WT) were anesthetized and perfused as described above. Brain tissue was embedded in paraffin and was cut into 20 μm sections with a cryostat. Sections were blocked in 3% normal goat serum and incubated overnight with antibody to torsinA (1:1000 diluted in PBS with 2.5% normal serum) and ubiquitin (1:1000) from DakoCytomation, Carpinteria CA. The torsinA antibody used was a rabbit polyclonal antibody to human torsinA that has been used previously in other studies [17; 80]. Immunolabeling was visualized with fluorochrome-conjugated secondary antibodies, Alexa488 and Alexa594 from Molecular Probes, Eugene, Oreg.

Results

The generation of the Dyt1 DGAG mice and their behavioral characteristics are described above. The present results section focuses on neurochemical and gross brain changes seen in this mouse model. Briefly reiterating the above results, at 3 months the mice did not deficits in behavioral performance on any of the battery of tests as compared to wild-type animals. At 6 months, neither the WT no eth Dyt1 DGAG showed any observable hindlimb extension or truncal acrhcing, and all mice showed a normal splaying of hindlimbs if suspended by the tail, as well as exhibiting strong righting reflexes if tipped on their side. Taken together, the beam walking and rotarod tests showed that the 6-month old Dyt1 DGAG mice displayed significant motor coordination and balance deficits in the beam walking test. The male Dyt1 DGAG mice also had an abnormal gait and open field data suggested that Dyt1 DGAG mice (particularly the male mice) displayed hyperactivity.

To determine if the observed hyperactivity and motor control deficits are correlated with abnormal dopaminergic mechanisms, the levels of striatal dopamine (DA) and DA metabolites, DOPAC and HVA were measured. No difference was detected in the amount of DA (P=0.29) and DOPAC (P=0.31) between mutant and wild-type male mice. There was, however, a 27% reduction in HVA in Dyt1 DGAG male mice (P=0.03). No difference was detected in the ratios of DOPAC to DA (P=0.51) and HVA to DA (P=0.17). Mutant and wild-type female mice did not differ in any of the measurements: DA (P=0.90), DOPAC (P=0.20), HVA (P=0.47), DOPAC to DA (P=0.51), and HVA to DA ratios (P=0.17).

To determine the effect of Dyt1 DGAG on the development of the brain, especially the basal ganglia system, the gross brain anatomy was examined via thionin-based Niss1 staining. The Niss1 staining revealed no obvious differences between Dyt1 DGAG mice and their WT littermates. Dyt1 DGAG mice showed well-developed brain structures. The size and weight of the Dyt1 DGAG brains were similar to their control counterparts. The corpus callosum was present in Dyt1 DGAG mice. The Dyt1 DGAG mice also showed well-developed cerebral cortex, hippocampus, and cerebellum.

The thickness and layers of the Dyt1 DGAG cerebral cortex appeared normal. The Dyt1 DGAG mice also had well developed hippocampus with CA1, CA2, CA3, and dentate gyrus. The cerebellums of the Dyt1 DGAG mice were also well developed with normal locations and packing density of Purkinje cells and granule cells. Using this Niss1 stain, the apparent size and density of the neurons in the basal ganglia circuits, the caudate-putamen and lateral globus pallidus, the medial globus pallidus, the subthalamic nucleus, and substantia nigra pars compacta and pars reticulata, were indistinguishable between WT and Dyt1 DGAG mice.

Since protein aggregates were found in the brain stem of dystonic patients, immunohistochemistry analysese were performed in order to determine if the mutant mice also have protein aggregates. In the brain of control animals, expression of torsinA occurred in many brain regions, including various nuclei of the pons, a finding which is consistent with previous studies [17; 19; 81; 82]. In male Dyt1 DGAG mice, there was a marked increase in the aggregation of this protein in the pontine nuclei. Notably, protein aggregates that stained for both torsinA and ubiquitin were present surrounding the nucleus in cells of the pontine nuclei. No aggregates were detected in the cortex, substantia nigra pars compacta, and other midbrain regions. Female Dyt1 DGAG and wild-type mice did not differ in their immunostaining for torsinA and ubiquitin. No increased protein aggregates were noted in female mutant mice.

Discussion

The gene-targeted mouse model of Dyt1 DGAG mimics the mutation found in DYT1 dystonic patients and the observed motor performance deficits and tissue aggregations indicate that the mouse model recapitulates some of the phenotypes seen in dystonic patients.

The motor behavioral characterization of Dyt1 DGAG mice showed that when one copy of the Dyt1 gene is mutated, fine motor balance and coordination are impaired. The consistent muscle contractions of dystonic patients can prevent them from smoothly coordinating movements and challenge their ability to maintain balance. The motor coordination and balance of Dyt1 DGAG mice were characterized using both the rotarod and beam-walking tests. Dyt1 DGAG mice performed normally on the rotarod. The results presented here differ from the rotarod deficit of motor learning that was detected in another mouse model that overexpressed human wild-type torsinA [83]. The difference may stem from the variability in the number of test trials. Our animals were tested for 2 days to determine motor coordination deficits, and the transgenic mice were tested for 5 days.

While the rotarod is traditionally a test of gross motor skills, the beam-walking test poses challenges to the subject's fine motor balance and coordination skills [84]. Dyt1 DGAG mice took the same time to cross the beams, but displayed a greater tendency to slip during the traversal. Two male mutant mice showed a more severe phenotype when they were unsuccessful in crossing and dropped from at least one of the small beams. This beam-walking test for imbalance and incoordination is sensitive to changes in dopaminergic function as well as aging in rodents [85; 86; 87]. Not surprisingly, some Parkinson's disease mouse models also exhibit beam-walking deficits similarly seen in Dyt1 DGAG mice [88; 89]. The commonality establishes yet another link between the two disorders which may be caused by the abnormal functioning of the dopaminergic system. Lewy bodies found in Parkinsonic patients have been shown to contain torsinA and alpha-synuclein in close association with each other [90]. In addition, primates injected with MPTP, known to cause dopaminergic neuronal cell death, expressed dystonic symptoms before the eventual Parkinsonic signs [91].

The second motor behavioral phenotype observed in Dyt1 DGAG mice is hyperactivity. Analysis of all parameters indicative of activity level including movement number and count, along with the reported total distance traveled and horizontal activity shows that Dyt1 DGAG mice have an increased level of activity. This heightened activity was also seen in the transgenic mice that carry an overexpression of human torsinA [42]. Hyperactivity has been detected also in mice that have alterations in their dopaminergic system, such as the dopamine receptor 3 knockout and dopamine transporter knockout mouse [92; 93; 94]. The latter provides compelling evidence that a hyperdopaminergic state can lead to a hyperactive phenotype [95]. Alternatively, hyperactivity can also be associated with increased sensitization of the neuronal system to dopamine, which has been shown in mice that were deprived of dopamine throughout development [96].

Dyt1 DGAG mice had a mild gait deficit expressed as an increase in overlap distance. Parkinsonic mice display decreases in stride distance which can be expected because of the short shuffling step typically seen in Parkinson's patients [97; 75]. The staggering movements of the R6/2 Huntington mice and gait that lacked normal step pattern corresponded to the abnormal gait of patients (Carter et al., 1999). With the large spectrum of phenotypic expression of dystonic patients, it is less clear what the gait of a dystonic mouse should look like. Since the overlap measurement is an indication of the degree of precision and coordination of the forepaw and hindpaws during walking [84; 69], the observed increase in overlap suggests that these mice have a detectable lack of precise coordination during movement.

When viewing all the motor deficits as a whole, a possible age-dependent severity in motor deficits can be seen in Dyt1 DGAG mice. A difference in beam-walking slip numbers between WT and mutant mice was apparent only in aged mice. Aging mechanisms, such as oxidative stress, could contribute to the expression of motor abnormalities. TorsinA expression has been shown to increase in mice when MPTP, an oxidative stress producing and neurodegenerative toxin, is administered [98]. In addition, if torsinA is neuroprotective, changes that accumulate over time in cells that lack that protection may eventually affect behavior at an older age.

While it is tempting to view a 6-month mouse (with an approximate 2-year lifespan) as equivalent to a human who has lived a quarter of his/her lifespan, the developmental progression between mice and human may not be proportionally paralleled. The potential difference in developmental progression may explain why the phenotype appears in 6-month old Dyt1 DGAG mice, when they are commonly and potentially mistakenly considered to be in their adulthood, while the phenotype normally appears during childhood or adolescence in human patients.

While dystonic patients display isolated overactive opposing muscles, twisting of limb, and repetitive movements [99], Dyt1 DGAG mice exhibited general hyperactivity and a deficient performance on a task that requires high motor coordination and balance. We propose these possibilities to understand the differences. First, the phenotype displayed in these mice may be the full expression of Dyt1 dystonia as it can appear in mice with the mutated torsinA given their distinct anatomy, physiology, and lifespan. Biochemical and developmental variation as well as differences in absolute rates of physiological processes may affect the replication of a human disorder in mice [100]. Also, the difference in developmental timeline between mouse and human may prevent the accurate modeling in mice of a progressive neurochemical disorder such as dystonia. The disease progression, which could lead to more severe symptoms, may require a longer time of aging that could exceed the lifespan of mice. This possibility has been noted in an analysis of a Parkinson mouse model [101].

The phenotype of Dyt1 DGAG mice may also represent a milder version of the phenotype detectable in patients with the penetrant mutation. The large variation in phenotypic presentation of early-onset dystonia has been well documented to range from tremors to excessive and lethal muscle contractions throughout the body [102; 103]. Also, the degree of symptomatic severity observed in the mutant mice may be influenced by their specific 129/SvJ, BALB/c, and C57BL/6 backgrounds. Alternatively, the motor deficits observed may largely mirror individuals who are clinically diagnosed as non-manifesting carriers. The DGAG mutation has an approximate 3040% penetrance [4]. The majority of carriers never express movement deficit symptoms. However, even in non-manifesting patients, increases in brain activity as detected by PET have been seen [104]. Perhaps unlike in non-manifesting carriers, in mice, the subtle neuronal circuitry change due to the mutated torsinA leads to measurable changes of motor behavior. If this is the case, these mice may be capable of expressing the severe muscle contractions seen in patients if the correct genetic modifier or environmental factor is introduced to the system.

To characterize the neurochemical changes caused by the presence of mutated torsinA, measurement of striatal dopamine and its metabolites of the mice showed a 27% decrease in striatal HVA content in only male Dyt1 DGAG mice. Interestingly, this decrease in HVA mirrors the decrease of this metabolite in dopa-responsive dystonic patients with a mutation in GTP cyclohydrolase [105]. Dopamine level and metabolic alterations have been shown in DYTI patients and the transgenic mouse model, including a decrease in dopamine content in striatal tissues and an increase in the ratio of DOPAC to dopamine [106; 107; 108]. Although discrepancies exist among these studies and our own data, they all point to the dopaminergic system as a possible site of alteration caused by the mutation in Dyt1. A recent in vitro study, has in fact shown that one of torsinA's role may be in regulating the activity of the dopamine transporter [36].

While abnormality in the gross brain anatomy was not observed in the Dyt1 DGAG mice, an immunohistochemical analysis showed that male Dyt1 DGAG mice have aggregates of ubiquitin and torsinA in the pontine nuclei, consistent with previous reports demonstrating the appearance of protein aggregates in the same brain areas in DYT1 dystonic patients and transgenic mice overexpressing mutant torsinA [109; 42]. These findings raise the possibility that the neuronal dysfunction in this brain area could contribute to motor deficits in the animals. One of the pontine nuclei, the pedunculopontine nucleus, is thought to regulate movement with input coming from the basal ganglia and output to several regions including the thalamus and subthalamic nucleus [reviewed in 110].

Damage to this region has been attributed to the development of Parkinson's [111; 112].

A sex-difference was noted in the behavioral manifestation of the mutation, as well as in DA and DA metabolite levels and protein aggregation in the brain. While abnormalities were noted in mutant male mice of all tests, mutant female mice were indistinguishable from their wild-type counterparts in all tests. This gender bias has been previously reported in patients as well. In one study, among carriers and non-carriers of DGAG patients clinically diagnosed with primary dystonia, males were found to have a significantly younger age of onset and more occurrences of generalized versus localized distribution of dystonia than female patients tested [3]. In another study, among the over 50 Jewish patients examined, 43.5% of female patients and only 23.1% of males were given a qualitatively “good” prognosis mark 10 years after onset [113].

Sex differences in the central nervous system of rodents have been substantially documented. For example, neurotoxicity from 1-methyl-4-phenyl-1,2,3,6-tetrahydropyridine and amphetamine-induced dopamine release in mice were shown to be significantly less in female than male mice [114; 115]. Estrogen and progesterone were demonstrated to be neuroprotective against these types of toxicity [114; 116]. In addition to hormonal differences, variations in several aspects of the CNS motor control regions were observed in female and male rodents. The substantia nigra pars reticulata of female mice has a higher level than male mice of GABA immunoreactivity and the al subunit mRNA of the GABA receptor [ 117]. Female rodents were also reported to have a more efficient recovery and vesicular packaging of extracellular DA and less pruning of striatal D1 and D2 receptors during periadolescence [118; 119]. Some of these differences may account for the difference in behavioral, neurochemical, and cellular phenotypes in our Dyt1 DGAG female and male mice.

In conclusion, mice carrying a DGAG Dyt1 allele were generated and characterized which mimic the mutation found in DYT1 dystonic patients. Only male Dyt1 DGAG mice displayed behavioral abnormalities, neurochemical, and cellular changes that can be associated with the dystonic phenotype, making it a relevant model with which to further study DYT1 dystonia. Furthermore, these findings have provided evidence to support changes in the dopaminergic system, which may be age- and sex-dependent, as a site for abnormalities in Dyt1 DGAG animals.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

The references cited herein throughout, to the extent that they provide exemplary procedural or other details supplementary to those set forth herein, are all specifically incorporated herein by reference. In certain of the above examples, articles are identified using a number in square brackets. The following is a listing of the references that correspond to those number.

[1] Rahman, Z., Gold, S. J., Potenza, M. N., Cowan, C. W., Ni, Y. G., He, W., Wensel, T. G. and Nestler, E. J. (1999) Cloning and characterization of RGS9-2: a striatal-enriched alternatively spliced product of the RGS9 gene, J Neurosci 19, 2016-26.

[2] Heiman, G. A., Ottman, R., Saunders-Pullman, R. J., Ozelius, L. J., Risch, N. J. and Bressman, S. B. (2004) Increased risk for recurrent major depression in DYT1 dystonia mutation carriers, Neurology 63, 631-7.

[3] Bressman, S. B. et al. (2000) The DYT1 phenotype and guidelines for diagnostic testing, Neurology 54, 1746-52.

[4] Fahn, S. (1991) The genetics of idiopathic torsion dystonia, Int J Neurol 25-26, 70-80.

[5] Ozelius, L. J. et al. (1997) Fine localization of the torsion dystonia gene (DYT1) on human chromosome 9q34: YAC map and linkage disequilibrium, Genome Res 7, 483-94.

[6] Ozelius, L. J. et al. (1997) The early-onset torsion dystonia gene (DYT1) encodes an ATP-binding protein, Nat Genet 17, 40-8.

[7] Risch, N. et al. (1995) Genetic analysis of idiopathic torsion dystonia in Ashkenazi Jews and their recent descent from a small founder population, Nat Genet 9, 152-9.

[8] Maniak, S., Sieberer, M., Hagenah, J., Klein, C. and Vieregge, P. (2003) Focal and segmental primary dystonia in north-western Germany--a clinico-genetic study, Acta Neurol Scand 107, 228-32.

[9] Sibbing, D. et al. (2003) Candidate gene studies in focal dystonia, Neurology 61, 1097-101.

[10] Leung, J. C. et al. (2001) Novel mutation in the TORIA (DYT1) gene in atypical early onset dystonia and polymorphisms in dystonia and early onset parkinsonism, Neurogenetics 3, 133-43.

[11] Klein, C. et al. (2002) Epsilon-sarcoglycan mutations found in combination with other dystonia gene mutations, Ann Neurol 52, 675-9.

[12] Zimprich, A. et al. (2001) Mutations in the gene encoding epsilon-sarcoglycan cause myoclonus-dystonia syndrome, Nat Genet 29, 66-9.

[13] Ozelius, L.J. et al. (1998) The gene (DYT1) for early-onset torsion dystonia encodes a novel protein related to the Clp protease/heat shock family, Adv Neurol 78, 93-105.

[14] Augood, S. J., Penney, J. B., Jr., Friberg, I. K., Breakefield, X. O., Young, A. B., Ozelius, L. J. and Standaert, D. G. (1998) Expression of the early-onset torsion dystonia gene (DYTI) in human brain, Ann Neurol 43, 669-73.

[15] Augood, S. J., Martin, D. M., Ozelius, L. J., Breakefield, X. O., Penney, J. B., Jr. and Standaert, D. G. (1999) Distribution of the mRNAs encoding torsinA and torsinB in the normal adult human brain, Ann Neurol 46, 761-9.

[16] Oberlin, S., Konakova, M., Pulst, S M., Chesselet M F. (2002) in: International Dystonia Conference, Atlanta, Ga.

[17] Shashidharan, P., Kramer, B. C., Walker, R. H., Olanow, C. W. and Brin, M. F. (2000) Immunohistochemical localization and distribution of torsinA in normal human and rat brain, Brain Res 853, 197-206.

[18] Xiao, J., Gong, S., Zhao, Y. and LeDoux, M. S. (2004) Developmental expression of rat torsinA transcript and protein, Brain Res Dev Brain Res 152, 47-60.

[19] Konakova, M., Huynh, D. P., Yong, W. and Pulst, S. M. (2001) Cellular distribution of torsin A and torsin B in normal human brain, Arch Neurol 58, 921-7.

[20] Kustedjo, K., Deechongkit, S., Kelly, J. W. and Cravatt, B. F. (2003) Recombinant expression, purification, and comparative characterization of torsinA and its torsion dystonia-associated variant Delta E-torsinA, Biochemistry 42, 15333-41.

[21] Liu, Z., Zolkiewska, A. and Zolkiewski, M. (2003) Characterization of human torsinA and its dystonia-associated mutant form, Biochem J 374, 117-22.

[22] Kamm, C., Boston, H., Hewett, J., Wilbur, J., Corey, D. P., Hanson, P. I., Ramesh, V. and Breakefield, X. O. (2004) The early onset dystonia protein torsinA interacts with kinesin light chain 1, J Biol Chem 279, 19882-92.

[23] Caldwell, G. A., Cao, S., Sexton, E. G., Gelwix, C. C., Bevel, J. P. and Caldwell, K. A. (2003) Suppression of polyglutamine-induced protein aggregation in Caenorhabditis elegans by torsin proteins, Hum Mol Genet 12, 307-19.

[24] Hewett, J. et al. (2000) Mutant torsinA, responsible for early-onset torsion dystonia, forms membrane inclusions in cultured neural cells, Hum Mol Genet 9, 1403-13.

[25] Kustedjo, K., Bracey, M. H. and Cravatt, B. F. (2000) Torsin A and its torsion dystonia-associated mutant forms are lumenal glycoproteins that exhibit distinct subcellular localizations, J Biol Chem 275, 27933-9.

[26] McLean, P. J., Kawamata, H., Shariff, S., Hewett, J., Sharma, N., Ueda, K., Breakefield, X. O. and Hyman, B. T. (2002) TorsinA and heat shock proteins act as molecular chaperones: suppression of alpha-synuclein aggregation, J Neurochem 83, 846-54.

[27] Kuner, R. et al. (2003) TorsinA protects against oxidative stress in COS-1 and PC12 cells, Neurosci Lett 350, 153-6.

[28] Hewett, J. et al. (2003) TorsinA in PC12 cells: localization in the endoplasmic reticulum and response to stress, J Neurosci Res 72, 158-68.

[29] Goodchild, R. E. and Dauer, W. T. (2004) Mislocalization to the nuclear envelope: An effect of the dystonia-causing torsinA mutation, Proc Natl Acad Sci U S A.

[30] Naismith, T.V., Heuser, J.E., Breakefield, X.O. and Hanson, P.I. (2004) TorsinA in the nuclear envelope, Proc Natl Acad Sci U S A 101, 7612-7.

[31] Bragg, D. C. et al. (2004) Perinuclear biogenesis of mutant torsin-A inclusions in cultured cells infected with tetracycline-regulated herpes simplex virus type 1 amplicon vectors, Neuroscience 125, 651-61.

[32] Gonzalez-Alegre, P. and Paulson, H. L. (2004) Aberrant cellular behavior of mutant torsinA implicates nuclear envelope dysfunction in DYT1 dystonia, J Neurosci 24, 2593-601.

[33] Augood, S. J. et al. (2002) Dopamine transmission in DYT1 dystonia: a biochemical and autoradiographical study, Neurology 59, 445-8.

[34] Perlmutter, J. S., Stambuk, M. K., Markham, J., Black, K. J., McGee-Minnich, L., Jankovic, J. and Moerlein, S. M. (1998) Decreased [18F]spiperone binding in putamen in dystonia, Adv Neurol 78, 161-8.

[35] Asanuma, K., Ma, Y., Okulski, J., Dhawan, V., Chaly, T., Carbon, M., Bressman, S. B. and Eidelberg, D. (2005.) Decreased striatal D2 receptor binding in non-manifesting carriers of the DYT1 dystonia mutation, Neurology 64, 347-9.

[36] Torres, G. E., Sweeney, A. L., Beaulieu, J. M., Shashidharan, P. and Caron, M. G. (2004) Effect of torsinA on membrane proteins reveals a loss of function and a dominant-negative phenotype of the dystonia-associated DeltaE-torsinA mutant, Proc Natl Acad Sci U S A 101, 15650-5.

[37] Misbahuddin, A., Placzek, M. R., Taanman, J. W., Gschmeissner, S., Schiavo, G., Cooper, J. M. and Warner, T. T. (2004) Mutant torsinA, which causes early-onset primary torsion dystonia, is redistributed to membranous structures enriched in vesicular monoamine transporter in cultured human SH-SY5Y cells, Mov Disord.

[38] Berardelli, A., Rothwell, J. C., Hallett, M., Thompson, P. D., Manfredi, M. and Marsden, C. D. (1998) The pathophysiology of primary dystonia, Brain 121 (Pt 7), 1195-212.

[39] Richter, A. and Loscher, W. (2000) Animal models of dystonia, Funct Neurol 15, 259-67.

[40] Ozelius, L. J. et al. (1999) The TOR1A (DYT1) gene family and its role in early onset torsion dystonia, Genomics 62, 377-84.

[41] Sharma, N., Bragg, D. C., Petravicz, J., Standaert, D. G. and Breakefield, X. O. (2005) in: Animal Models of Movement Disorders, pp. 287-292 (Le Doux, M., Ed.) Elsevier Academic Press, Amersterdam.

[42] Shashidharan, P. et al. (2005) Transgenic mouse model of early-onset DYT1 dystonia, Hum Mol Genet 14, 125-33.

[43] LeDoux, M. S., Lorden, J. F. and Ervin, J. M. (1993) Cerebellectomy eliminates the motor syndrome of the genetically dystonic rat, Exp Neurol 120, 302-10.

[44] LeDoux, M. S., Lorden, J. F. and Meinzen-Derr, J. (1995) Selective elimination of cerebellar output in the genetically dystonic rat, Brain Res 697, 91-103.

[45] LeDoux, M. S. and Lorden, J. F. (2002) Abnormal spontaneous and harmaline-stimulated Purkinje cell activity in the awake genetically dystonic rat, Exp Brain Res 145, 457-67.

[46] Pizoli, C. E., Jinnah, H. A., Billingsley, M. L. and Hess, E. J. (2002) Abnormal cerebellar signaling induces dystonia in mice, J Neurosci 22, 7825-33.

[47] Grusser-Comehls, U., Grusser, C. and Baurle, J. (1999) Vermectomy enhances parvalbumin expression and improves motor performance in weaver mutant mice: an animal model for cerebellar ataxia, Neuroscience 91, 315-26.

[48] Grusser, C. and Grusser-Comehls, U. (1998) Improvement in motor performance of Weaver mutant mice following lesions of the cerebellum, Behav Brain Res 97, 189-94.

[49] Campbell, D. B., North, J. B. and Hess, E. J. (1999) Tottering mouse motor dysfunction is abolished on the Purkinje cell degeneration (pcd) mutant background, Exp Neurol 160, 268-78.

[50] LeDoux, M. S., Rutledge, S. L., Mountz, J. M. and Darji, J. T. (1995) SPECT abnormalities in generalized dystonia, Pediatr Neurol 13, 5-10.

[51] Carbon, M., Su, S., Dhawan, V., Raymond, D., Bressman, S. and Eidelberg, D. (2004) Regional metabolism in primary torsion dystonia: effects of penetrance and genotype, Neurology 62, 1384-90.

[52] Ventura, A., Meissner, A., Dillon, C. P., McManus, M., Sharp, P. A., Van Parijs, L., Jaenisch, R. and Jacks, T. (2004) Cre-lox-regulated conditional RNA interference from transgenes, Proc Natl Acad Sci U S A 101, 10380-5.

[53] Tiscomia, G., Tergaonkar, V., Galimi, F. and Verma, I. M. (2004) CRE recombinase-inducible RNA interference mediated by lentiviral vectors, Proc Natl Acad Sci U S A 101, 7347-51.

[54] Mandel, R. J. and Burger, C. (2004) Clinical trials in neurological disorders using AAV vectors: promises and challenges, Curr Opin Mol Ther 6, 482-90.

[55] Xia, H. et al. (2004) RNAi suppresses polyglutamine-induced neurodegeneration in a model of spinocerebellar ataxia, Nat Med 10, 816-20.

[56] Harper, S. Q. et al. (2005) RNA interference improves motor and neuropathological abnormalities in a Huntington's disease mouse model, Proc Natl Acad Sci U S A 102, 5820-5.

[57] Schwenk, F., Baron, U. and Rajewsky, K. (1995) A cre-transgenic mouse strain for the ubiquitous deletion of loxP-flanked gene segments including deletion in germ cells, Nucleic Acids Res 23, 5080-1.

[58] Granneman, J. G., Zhai, Y., Zhu, Z., Bannon, M. J., Burchett, S. A., Schmidt, C. J., Andrade, R. and Cooper, J. (1998) Molecular characterization of human and rat RGS 9L, a novel splice variant enriched in dopamine target regions, and chromosomal localization of the RGS 9 gene, Mol Pharmacol 54, 687-94.

[59] Thomas, E. A., Danielson, P. . and Sutcliffe, J. G. (1998) RGS9: a regulator of G-protein signalling with specific expression in rat and mouse striatum, J Neurosci Res 52, 118-24.

[60] Usui, H., Falk, J. D., Dopazo, A., de Lecea, L., Erlander, M. G. and Sutcliffe, J. G. (1994) Isolation of clones of rat striatum-specific mRNAs by directional tag PCR subtraction, J Neurosci 14, 4915-26.

[61] Jin, X. L., Guo, H., Mao, C., Atkins, N., Wang, H., Avasthi, P. P., Tu, Y. T. and Li, Y. (2000) Emx 1-specific expression of foreign genes using “knock-in” approach, Biochem Biophys Res Commun 270, 978-82.

[62] Guo, H., Hong, S., Jin, X. L., Chen, R. S., Avasthi, P. P., Tu, Y. T., Ivanco, T. L. and Li, Y. (2000) Specificity and efficiency of Cre-mediated recombination in Emx1-Cre knock-in mice, Biochem Biophys Res Commun 273, 661-5.

[63] Barski, J. J., Dethleffsen, K. and Meyer, M. (2000) Cre recombinase expression in cerebellar Purkinje cells, Genesis 28, 93-8.

[64] Kuligina, E., Lebedev, A. A. and Luchnikova, E. M. (1997) [A comparative genetic analysis of the role of the brain dopaminergic systems in the regulation of behavioral elements in the “open field” test in DBA/2J and C57BL/6J mice], Genetika 33, 1529-33.

[65] Deutsch, S. I., Rosse, R. B., Paul, S. M., Riggs, R. L. and Mastropaolo, J. (1997) Inbred mouse strains differ in sensitivity to “popping” behavior elicited by MK-801, Pharmacol Biochem Behav 57, 315-7.

[66] Laghmouch, A., Bertholet, J. Y. and Crusio, W. E. (1997) Hippocampal morphology and open-field behavior in Mus musculus domesticus and Mus spretus inbred mice, Behav Genet 27, 67-73.

[67] Gerlai, R. (1996) Gene-targeting studies of mammalian behavior: is it the mutation or the background genotype?, Trends Neurosci 19, 177-81.

[68] Crawley, J. N. et al. (1997) Behavioral phenotypes of inbred mouse strains: implications and recommendations for molecular studies, Psychopharmacology (Berl) 132, 107-24.

[69] Dunnett, S. B. (2003) in: Mouse Behavioral Phenotyping Short Course (Crawley, J., Ed.) Society for Neuroscience, New Orleans.

[70] Whitfield, H. J., Jr., Brady, L. S., Smith, M. A., Mamalaki, E., Fox, R.J. and Herkenham, M. (1990) Optimization of cRNA probe in situ hybridization methodology for localization of glucocorticoid receptor mRNA in rat brain: a detailed protocol, Cell Mol Neurobiol 10, 145-57.

[71] Li, Y., Erzurumlu, R. S., Chen, C., Jhaveri, S. and Tonegawa, S. (1994) Whisker-related neuronal patterns fail to develop in the trigeminal brainstem nuclei of NMDAR1 knockout mice, Cell 76, 427-37.

[72] Oberdick, J., Schilling, K., Smeyne, R. J., Corbin, J. G., Bocchiaro, C. and Morgan, J. I. (1993) Control of segment-like patterns of gene expression in the mouse cerebellum, Neuron 10, 1007-18.

[73] Alisky, J. M., Hughes, S. M., Sauter, S. L., Jolly, D., Dubensky, T. W., Jr., Staber, P. D., Chiorini, J. A. and Davidson, B. L. (2000) Transduction of murine cerebellar neurons with recombinant FIV and AAV5 vectors, Neuroreport 11, 2669-73.

[74] Soriano, P. (1999) Generalized lacZ expression with the ROSA26 Cre reporter strain, Nat Genet 21, 70-1.

[75] Fleming, S. and Chesselet, M. -F. (2005) in: Animal Models of Movement Disorders, pp. 183-192 (LeDoux, M., Ed.) Elsevier Academic Press, Burlington, Mass.

[76] Diaz, S. L., Kemmling, A. K., Balerio, G. N., 2003. Baclofen reestablishes striatal and cortical dopamine concentrations during naloxone-precipitated withdrawal. Neurochem. Int. 42, 293-298.

[77] Mandavilli, B. S., Ali, S. F., Van Houten, B., 2000. DNA damage in brain mitochondria caused by aging and MPTP treatment. Brain Res. 885, 45- 52.

[78] Perez, V., Unzeta, M., 2003. PF 9601N [N-(2-propynyl)-2-(5-benzyloxyindolyl) methylamine], a new MAO-B inhibitor, attenuates MPTP-induced depletion of striatal dopamine levels in C57/BL6 mice. Neurochem. Int. 42, 221- 229.

[79] Guo, H., Christoff, J. M., Campos, V. E., Jin, X. L., Li, Y., 2000. Normal corpus callosum in Emx1 mutant mice with C57BL/6 background. Biochem. Biophys. Res. Commun. 276, 649- 653.

[80] Shashidharan, P., Sandu, D., Potla, U., Armata, I. A., Walker, R. H., McNaught, K. S., Weisz, D., Sreenath, T., Brin, M. F., Olanow, C. W., 2005. Transgenic mouse model of early-onset DYT1 dystonia. Hum. Mol. Genet. 14, 125-133

[81] Rostasy, K., Augood, S. J., Hewett, J. W., Leung, J. C., Sasaki, H., Ozelius, L. J., Ramesh, V., Standaert, D. G., Breakefield, X. O., Hedreen, J. C., 2003. TorsinA protein and neuropathology in early onset generalized dystonia with GAG deletion. Neurobiol. Dis. 12, 11-24.

[82] Walker, R. H., Brin, M. F., Sandu, D., Gujjari, P., H of, P. R., Warren Olanow, C., Shashidharan, P., 2001. Distribution and immunohistochemical characterization of torsinA immunoreactivity in rat brain. Brain Res. 900, 348-354.

[83] Sharma, N., Baxter, M. G., Petravicz, J., Bragg, D. C., Schienda, A., Standaert, D. G., Breakefield, X. O., 2005. Impaired motor learning in mice expressing torsinA with the DYT1 dystonia mutation. J. Neurosci. 25, 5351-5355.

[84] Carter, R. J., Lione, L. A., Humby, T., Mangiarini, L., Mahal, A., Bates, G. P., Dunnett, S. B., Morton, A. J., 1999. Characterization of progressive motor deficits in mice transgenic for the human Huntington's disease mutation. J. Neurosci. 19, 3248-3257.

[85] Dluzen, D. E., Liu, B., Chen, C. Y., DiCarlo, S. E., 1995. Daily spontaneous running alters behavioral and neurochemical indexes of nigrostriatal function. J. Appl. Physiol. 78, 1219-1224.

[86] Dluzen, D. E., Gao, X., Story, G. M., Anderson, L. I., Kucera, J., Walro, J. M., 2001. Evaluation of nigrostriatal dopaminergic function in adult +/+ and +/□ BDNF mutant mice. Exp. Neurol. 170, 121-128.

[87] Drucker-Colin, R., Garcia-Hemandez, F., 1991. A new motor test sensitive to aging and dopaminergic function. J. Neurosci. Methods 39, 153-161.

[88] Goldberg, M. S., Fleming, S. M., Palacino, J. J., Cepeda, C., Lam, H. A., Bhatnagar, A., Meloni, E. G., Wu, N., Ackerson, L. C., Klapstein, G. J., Gajendiran, M., Roth, B. L., Chesselet, M. F., Maidment, N. T., Levine, M. S., Shen, J., 2003. Parkin-deficient mice exhibit nigrostriatal deficits but not loss of dopaminergic neurons. J. Biol. Chem. 278, 43628-43635.

[89] Fleming, S. M., Salcedo, J., Femagut, P. O., Rockenstein, E., Masliah, E., Levine, M. S., Chesselet, M. F., 2004. Early and progressive sensorimotor anomalies in mice overexpressing wild-type human alpha-synuclein. J. Neurosci. 24, 9434-9440.

[90] Sharma, N., Hewett, J., Ozelius, L. J., Ramesh, V., McLean, P. J., Breakefield, X. O., Hyman, B. T., 2001. A close association of torsinA and alphasynuclein in Lewy bodies: a fluorescence resonance energy transfer study. Am. J. Pathol. 159, 339-344.

[91] Perlmutter, J. S., Tempel, L. W., Black, K. J., Parkinson, D., Todd, R. D., 1997. MPTP induces dystonia and parkinsonism. Clues to the pathophysiology of dystonia. Neurology 49, 1432-1438.

[92] Accili, D., Fishbum, C. S., Drago, J., Steiner, H., Lachowicz, J. E., Park, B. H., Gauda, E. B., Lee, E. J., Cool, M. H., Sibley, D. R., Gerfen, C. R., Westphal, H., Fuchs, S., 1996. A targeted mutation of the D3 dopamine receptor gene is associated with hyperactivity in mice. Proc. Natl. Acad. Sci. U. S. A. 93, 1945-1949.

[93] Xu, M., Koeltzow, T. E., Santiago, G. T., Moratalla, R., Cooper, D. C., Hu, X. T., White, N. M., Graybiel, A. M., White, F. J., Tonegawa, S., 1997. Dopamine D3 receptor mutant mice exhibit increased behavioral sensitivity to concurrent stimulation of D1 and D2 receptors. Neuron 19, 837-848.

[94] Zhuang, X., Oosting, R. S., Jones, S. R., Gainetdinov, R. R., Miller, G. W., Caron, M. G., Hen, R., 2001. Hyperactivity and impaired response habituation in hyperdopaminergic mice. Proc. Natl. Acad. Sci. U. S. A. 98, 1982-1987.

[95] Viggiano, D., Ruocco, L. A., Sadile, A. G., 2003. Dopamine phenotype and behaviour in animal models: in relation to attention deficit hyperactivity disorder. Neurosci. Biobehav. Rev. 27, 623-637.

[96] Kim, D. S., Szczypka, M. S., Palmiter, R. D., 2000. Dopamine-deficient mice are hypersensitive to dopamine receptor agonists. J. Neurosci. 20, 4405-4413.

[97] Femagut, P. O., Diguet, E., Labattu, B., Tison, F., 2002b. A simple method to measure stride length as an index of nigrostriatal dysfunction in mice. J. Neurosci. Methods 113, 123-130.

[98] Kuner, R., Teismann, P., Trutzel, A., Naim, J., Richter, A., Schmidt, N., Bach, A., Ferger, B., Schneider, A., 2004. TorsinA, the gene linked to early-onset dystonia, is upregulated by the dopaminergic toxin MPTP in mice. Neurosci. Lett. 355, 126-130.

[99] Fahn, S., 1988. Concept and classification of dystonia. Adv. Neurol. 50, 1-8.

[100] Erickson, R. P., 1989. Why isn't a mouse more like a man? Trends Genet. 5, 1-3.

[101] Shimohama, S., Sawada, H., Kitamura, Y., Taniguchi, T., 2003. Disease model: Parkinson's disease. Trends Mol. Med. 9, 360-365.

[102] Bentivoglio, A. R., Loi, M., Valente, E. M., Ialongo, T., Tonali, P., Albanese, A., 2002. Phenotypic variability of DYT1-PTD: does the clinical spectrum include psychogenic dystonia? Mov. Disord. 17, 1058-1063.

[103] Opal, P., Tintner, R., Jankovic, J., Leung, J., Breakefield, X. O., Friedman, J., Ozelius, L., 2002. Intrafamilial phenotypic variability of the DYT1 dystonia: from asymptomatic TOR1A gene carrier status to dystonic storm. Mov. Disord. 17, 339-345.

[104] Eidelberg, D., Moeller, J. R., Antonini, A., Kazumata, K., Nakamura, T., Dhawan, V., Spetsieris, P., deLeon, D., Bressman, S. B., Fahn, S., 1998. Functional brain networks in DYT1 dystonia. Ann. Neurol. 44, 303-312.

[105] Hyland, K., Munk-Martin, G. R., Arnold, T. L., Engle, L. A., 2003. The hph-1 mouse: a model for dominantly inherited GTP-cyclohydrolase deficiency. Ann. Neurol. 54, S46-S48.

[106] Augood, S. J., Hollingsworth, Z., Albers, D. S., Yang, L., Leung, J. C., Muller, B., Klein, C., Breakefield, X. O., Standaert, D. G., 2002. Dopamine transmission in DYT1 dystonia: a biochemical and autoradiographical study. Neurology 59, 445-448.

[107] Furukawa, Y., Hornykiewicz, O., Fahn, S., Kish, S. J., 2000. Striatal dopamine in early-onset primary torsion dystonia with the DYTI mutation. Neurology 54, 1193-1195.

[108] Shashidharan, P., Brin, M. F., Gujjari, D., Sandu, D., Olanow, C., 2002. A transgenic mouse model for DYTI Dystonia. 4th International Dystonia Symposium.

[109] McNaught, K. S., Kapustin, A., Jackson, T., Jengelley, T. A., Jnobaptiste, R., Shashidharan, P., Perl, D. P., Pasik, P., Olanow, C. W., 2004. Brainstem pathology in DYT1 primary torsion dystonia. Ann. Neurol. 56, 540-547.

[110] Mena-Segovia, J., Bolam, J. P., Magill, P. J., 2004. Pedunculopontine nucleus and basal ganglia: distant relatives or part of the same family? Trends Neurosci. 27, 585-588.

[111] Pahapill, P. A., Lozano, A. M., 2000. The pedunculopontine nucleus and Parkinson's disease. Brain 123 (Pt. 9), 1767-1783.

[112] Zweig, R. M., Jankel, W. R., Hedreen, J. C., Mayeux, R., Price, D. L., 1989. The pedunculopontine nucleus in Parkinson's disease. Ann. Neurol. 26, 41-46.

[113] Inzelberg, R., Kahana, E., Korczyn, A. D., 1988. Clinical course of idiopathic torsion dystonia among Jews in Israel. Adv. Neurol. 50, 93-100.

[114] Dluzen, D. E., McDermott, J. L., Liu, B., 1996. Estrogen alters MPTP-induced neurotoxicity in female mice: effects on striatal dopamine concentrations and release. J. Neurochem. 66, 658- 666.

[115] Freyaldenhoven, T. E., Cadet, J. L., Ali, S. F., 1996. The dopamine-depleting effects of 1-methyl-4-phenyl-1,2,3,6-tetrahydropyridine in CD-1 mice are gender-dependent. Brain Res. 735, 232-238.

[116] Yu, L., Liao, P. C., 2000. Estrogen and progesterone distinctively modulate methamphetamine-induced dopamine and serotonin depletions in C57BL/6J mice. J. Neural Transm. 107, 1139-1147.

[117] Ravizza, T., Friedman, L. K., Moshe, S. L., Veliskova, J., 2003. Sex differences in GABA(A)ergic system in rat substantia nigra pars reticulata. Int. J. Dev. Neurosci. 21, 245-254.

[118] Anderson, S. L., Teicher, M. H., 2000. Sex differences in dopamine receptors and their relevance to ADHD. Neurosci. Biobehav. Rev. 24, 137-141.

[119] Bhatt, S. D., Dluzen, D. E., 2005. Dopamine transporter function differences between male and female CD-1 mice. Brain Res. 1035, 188-195.

We claim:

1. A method for treating a neuronal disease in a mammal comprising selectively down-regulating the expression and/or activity of wild-type Dyt1 in Purkinje cells of said mammal.

2. The method of claim 1, wherein said selectively down-regulating wild-type Dyt1 in Purkinje cells comprises administering to the mammal an expression construct that comprises a Purkinje cell-specific promoter operably linked to a nucleic acid that inhibits the expression of wild-type Dyt1.

3. The method of claim 2, wherein said expression construct comprises a viral vector.

4. The method of claim 3, wherein said viral vector is an adenoassociated viral vector.

5. The method of claim 3, wherein said viral vector comprises a polynucleotide sequence of about 8 to 80 nucleotides in length targeted to a nucleic acid molecule encoding DYT1, wherein said polynucleotide of 8 to 80 nucleotides specifically hybridizes in Purkinje cells with a nucleic acid molecule the encodes DYT1 and inhibits the expression of DYT1 in said Purkinje cells.

6. The method of claim 5, wherein said nucleic a polynucleotide sequence of about 8 to 80 nucleotides in length targeted to a nucleic acid molecule encoding DYTI is a polynucleotide of about 15 to about 30 nucleotides in length.

7. The method of claim 5, wherein said polynucleotide sequence of about 8 to 80 nucleotides in length targeted to a nucleic acid molecule encoding DYT1 is a polynucleotide of about 20 to about 25 nucleotides in length.

8. The method of claim 2, wherein said expression construct is administered systemically.

9. The method of claim 2, wherein said expression construct is administered via an intrathecal catheter.

10. The method of claim 2, wherein said expression construct is administered via intracerebellar injection.

11. The method of claim 2, wherein said expression construct is administered in combination with at least one additional drug that is used for the treatment of dystonia or related tremor disorders.

12. The method of claim 1, wherein said subject is a human subject.

13. The method of claim 1, wherein said neuronal disorder is selected from the group consisting of a motor deficient disorder, a neurodegenerative disease, a neurodevelopmental disorder and a neurophyschiatric disease.

14. The method of claim 13, wherein said neuronal disorder is dystonia, Parkinson's disease or Huntington's disease.

15. The method of claim 14, wherein said dystonia is Parkinson's disease-related dystonia.

16. The method of claim 1, wherein said expression is inhibited by at least 40% as measured by a suitable assay.

17. The method of claim 2, wherein said expression construct comprises a duplexed antisense compound comprising a polynucleotide sequence of 8 to 80 nucleotides in length targeted to a nucleic acid molecule encoding Dyt1 with at least one natural or modified nucleobase forming an overhang at a terminus of said sequence; and (b) the complementary sequence of said sequence (a) having optionally at least one natural or modified nucleobase forming an overhang at a terminus of said complementary sequence; wherein said sequences (a) and (b), when hybridized, have at least one single-stranded overhang and at least one of terminus of said hybridized duplex, and wherein said duplex when interacted with a nucleic acid molecule encoding said Dyt 1 will inhibit expression of TorsinA in Purkinje cells.

18. The method of claim 17, wherein said polynucleotide specifically hybridizes to a sequence of said Dyt1 within at least 8 to 80 nucleotides extending 5′ of nucleic acid 645 of SEQ ID NO: 1, 5′ of nucleic acid 719 of SEQ ID NO: 1, 5′ of nucleic acid 793 of SEQ ID NO: 1, 5′ of nucleic acid 969 of SEQ ID NO: 1, 5′ of nucleic acid 1334, or 5′ of nucleic acid 1439 of SEQ ID NO: 1.

19. The method of claim 18, wherein said sequence specifically hybridizes with nucleic acids 625 to 645 of SEQ ID NO: 1, 686 to 719 of SEQ ID NO: 1, 772 to 793 of SEQ ID NO: 1, 931 to 969 of SEQ ID NO:1, 1299 to 1334 of SEQ ID NO: 1 or 1419 to 1439 of SEQ ID NO: 1.

20. A method for treating dystonia comprising inhibiting expression of a Dyt1 in Purkinje cells comprising: contacting a cell expressing a Dyt1 with a double stranded RNA comprising a sequence capable of hybridizing to Dyt1 mRNA corresponding to the polynucleotide sequences of SEQ ID NOS: 3-14, in an amount sufficient to elicit RNA interference; and inhibiting expression of the Dyt1 gene in the Purkinje cell.

21. The method of claim 20, wherein the double stranded RNA is provided by introducing a short interfering RNA (siRNA) into the cell by a method selected from the group consisting of transfection, electroporation, and microinjection.

22. The method of claim 20, wherein the double stranded RNA is provided by introducing a short interfering RNA (siRNA) into the cell by an expression vector.

23. The method of claim 22, wherein said expression vector comprises a Purkinje specific promoter operatively linked to said siRNA.

24. The method of claim 23, wherein said promoter is a Pcp2 promoter.

25. The method of claim 22, wherein said expression vector is a viral expression vector.

26. The method of claim 25, wherein said viral expression vector is an adenoassociated viral vector.

27. The method of claim 1, wherein said method provides an improved motor coordination in said mammal.

28. The method of claim 1, wherein said method provides an improved balance in said mammal.


SEQUENCE LISTING (15 sequences)

SEQ ID NO: 1 - DNA, 2597 bp, Homo sapiens
  misc_feature (13)..(13): n is a, c, g, or t
  misc_feature (86)..(86): n is a, c, g, or t
  misc_feature (137)..(137): n is a, c, g, or t
  misc_feature (283)..(283): n is a, c, g, or t
  misc_feature (293)..(293): n is a, c, g, or t
  misc_feature (329)..(329): n is a, c, g, or t
  misc_feature (335)..(335): n is a, c, g, or t
  misc_feature (493)..(493): n is a, c, g, or t
ctgaaaatagctncttattattattattattattattattatttgcgggagggagcacagtcttgctctgtctcccaggctggagntgcagtggtgagatctcggctcactgcaatctccgcctcctgggttcaagngcagttgctcatgtgtcagcctccccagtagctagggctacaggtgcctaccaccacaccggctaattttatatttttagtagagacgtggtttcaccatgttggtcaggctggtctcgaactcctgacctcaggtgatccgcccncctcagcctncccaaagggctgggattacaggcaggagccaccatncctggnaaaaataacgtccataaacaaaaacacgtggccaacagggcggagcagaaccgagtttccggaagcaaaacagggctttgtaccgaacaaagatggcggccgccggcgtcgggaggagggctgccctgaagaaagatggcctccgcgagaggaggaanccggaagcgtgggtctggcggctgcaccggttcgcggtcggcgcgagaacaagcagggtggcgcgggtccgggcatgaagctgggccgggccgtgctgggcctgctgctgctggcgccgtccgtggtgcaggcggtggagcccatcagcctgggactggccctggccggcgtcctcaccggctacatctacccgcgtctctactgcctcttcgccgagtgctgcgggcagaagcggagccttagccgggaggcactgcagaaggatctggacgacaacctctttggacagcatcttgcaaagaaaatcatcttaaatgccgtgtttggtttcataaacaacccaaagcccaagaaacctctcacgctctccctgcacgggtggacaggcaccggcaaaaatttcgtcagcaagatcatcgcagagaatatttacgagggtggtctgaacagtgactatgtccacctgtttgtggccacattgcactttccacatgcttcaaacatcaccttgtacaaggatcagttacagttgtggattcgaggcaacgtgagtgcctgtgcgaggtccatcttcatatttgatgaaatggataagatgcatgcaggcctcatagatgccatcaagcctttcctcgactattatgacctggtggatggggtctcctaccagaaagccatgttcatatttctcagcaatgctggagcagaaaggatcacagatgtggctttggatttctggaggagtggaaagcagagggaagacatcaagctcaaagacattgaacacgcgttgtctgtgtcggttttcaataacaagaacagtggcttctggcacagcagcttaattgaccggaacctcattgattattttgttcccttcctccccctggaatacaaacacctaaaaatgtgtatccgagtggaaatgcagtcccgaggctatgaaattgatgaagacattgtaagcagagtggctgaggagatgacatttttccccaaagaggagagagttttctcagataaaggctgcaaaacggtgttcaccaagttagattattactacgatgattgacagtcatgattggcagccggagtcactgcctggagttggaaaagaaacaacactcagtccttccacacttccacccccagctcctttccctggaagaggaatccagtgaatgttcctgtttgatgtgacaggaattctccctggcattgtttccaccccctggtgcctgcaggccacccagggaccacgggcgaggacgtgaagcctcccgaacacgcacagaaggaaggagccagctcccagcccactcatcgcagggctcatgattttttacaaattatgttttaattccaagtgtttctgtttcaaggaaggatgaataagttttattgaaaatgtggtaactttatttaaaatgatttttaacattatgagagactgctcagattctaagttgttggccttgtgtgtgtgtttttttttaagttctcatcattattacatagactgtgaagtatctttactggaaatgagcccaagcacacatgcatggcatttgttcctgaacaggagggcatccctggggatgtggctggagcatgagccagctctgtcccaggatggtcccagcggatgctgccaggggcagtgaagtgtttaggtgaaggacaagtaggtaagaggacgccttcaggcaccacagataagcctgaaacagcctctccaagggttttcaccttagcaacaatgggagctgtgggagtgattttggccacactgtcaacatttgttagaaccagtcttttgaaagaaaagtatttccaacttgtcacttgccagtcactccgttttgcaaaaggtggcccttcactgtccattccaaatagcccacacgtgctctctgctggattctaaattatgtgaattttgccatattaaatcttcctcatttatactattatttgttacgttcaatcagaatccccgaaacctcctataaagcttagctgccccttctgaggatgctgagaacggtgtctttctttataaatgcaaatggctaccgttttacaataaaattttgcatgtgc

SEQ ID NO: 2 - protein, 332 aa, Homo sapiens
MKLGRAVLGLLLLAPSVVQAVEPISLGLALAGVLTGYIYPRLYCLFAECCGQKRSLSREALQKDLDDNLFGQHLAKKIILNAVFGFINNPKPKKPLTLSLHGWTGTGKNFVSKIIAENIYEGGLNSDYVHLFVATLHFPHASNITLYKDQLQLWIRGNVSACARSIFIFDEMDKMHAGLIDAIKPFLDYYDLVDGVSYQKAMFIFLSNAGAERITDVALDFWRSGKQREDIKLKDIEHALSVSVFNNKNSGFWHSSLIDRNLIDYFVPFLPLEYKHLKMCIRVEMQSRGYEIDEDIVSRVAEEMTFFPKEERVFSDKGCKTVFTKLDYYYDD

SEQ ID NO: 3 - DNA, 20 bp, Artificial sequence
  Synthetic primer
caggcttctggcacagcagc

SEQ ID NO: 4 - DNA, 21 bp, Artificial sequence
  Synthetic primer
cagagtggctgaggagatgac

SEQ ID NO: 5 - DNA, 18 bp, Artificial sequence
  Synthetic primer
cagagtggctgagatgac

SEQ ID NO: 6 - DNA, 20 bp, Artificial sequence
  Synthetic primer
ctgagatgacatttttcccc

SEQ ID NO: 7 - DNA, 23 bp, Artificial sequence
  Synthetic primer
gagtggctgagatgacatttttc

SEQ ID NO: 8 - RNA, 21 nt, Artificial sequence
  Synthetic primer
caggcugaugggcuccaccgc

SEQ ID NO: 9 - RNA, 25 nt, Artificial sequence
  Synthetic primer
acucggcgaagaggcaguagagacg

SEQ ID NO: 10 - RNA, 24 nt, Artificial sequence
  Synthetic primer
ccgcagcacucggcgaagaggcag

SEQ ID NO: 11 - RNA, 22 nt, Artificial sequence
  Synthetic primer
uuugcaagaugcuguccaaaga

SEQ ID NO: 12 - RNA, 25 nt, Artificial sequence
  Synthetic primer
agaagccacuguucuuguuauugaa

SEQ ID NO: 13 - RNA, 25 nt, Artificial sequence
  Synthetic primer
gcugcugugccagaagccacuguuc

SEQ ID NO: 14 - RNA, 15 nt, Artificial sequence
  Synthetic primer
uucauagccucggga

SEQ ID NO: 15 - DNA, 1397 bp, Mus musculus
cccacgcgtccggtgctgggcgcgcaaggtgcgcgggtccggttatgaagcttggccgggccgctctggccctgctgctgctggcgccgtgcgtggttcgtgcggtggagcccatcagcctgagtctggccctggccggcgtactcaccacctatatctcctaccctcgtctctactgcctcttcgccgagtgctgcggccagatgcggagcctcagccgggaggcgctgcagaaagatctggataacaagctctttggacagcatcttgcaaaaaaagtcatcctaaacgccgtgtctggtttcctaagcaacccgaagcccaagaagccccttaccctctctctgcacgggtggacgggcaccggcaaaaacttcgccagcaagatcatcgcggagaatatttacgagggcggactgaacagtgactatgtacacctgtttgtggccacgctacacttcccccacgcctctaacatcacacagtataaggaccagttacagatgtggatcagaggcaacgtgagcgcctgtgctcgctccatcttcatctttgatgagatggacaagatgcatgccggcctcatcgacgccatcaagcctttcctagactattacgatgtggtagatgaggtctcctatcagaaagccatcttcatcttcctcagcaatgcaggggcagagaggatcacagacgtggctctggatttctggaaaagtgggaagcagagggaagaaatcaagctcagagacatggagcccgccctggccgtgtcggtcttcaataacaagaacagtggcttctggcacagcagcctcattgaccggaacctcatagattattttgtccccttcctgcccctggagtacaagcacctgaaaatgtgcatcagagtggagatgcagtcccgaggctatgaagtagatgaggacatcatcagcaaggtagcggaagagatgacgttcttccccaaggaggagaaggtcttctctgacaagggctgcaagactgtgttcaccaagctggactactacctggatgactgaggccggctgccagggctgtgtcagtccgctcctccccagaagaggcttggacacttcctgctgaggttagcgggagaatcctgtttctgctcctcagagcctgcaggtgaccgcgctgtagggaccacagatgcaacatggacctgaagctcctggagcctccagagatgaaagcagctcccagcattctggacttgtgatttttttaaaattatgttttaatttcaggtgtttctgttttgaggcagaacaagtaagttttactgcaaatgtgataacgttttatttaaaatggtttttaatactataagaagctttcaaaaaaaaaaaaaaaaaaaaaaaaaaaaaaaa